(19) United States
(12) Patent Application Publication
(10) Pub. No.: US 2026/0013394 A1
(43) Pub. Date: Jan. 8, 2026

(54) COMPOUND, MATERIAL FOR ORGANIC ELECTROLUMINESCENCE DEVICE, ORGANIC ELECTROLUMINESCENCE DEVICE, AND ELECTRONIC APPARATUS

(71) Applicant: IDEMITSU KOSAN CO.,LTD., Tokyo (JP)

(72) Inventors: Ryota TAKAHASHI, Chiyoda-ku, Tokyo (JP); Sayaka MIZUTANI, Chiyoda-ku, Tokyo (JP); Masato MITANI, Chiyoda-ku, Tokyo (JP)

(73) Assignee: IDEMITSU KOSAN CO.,LTD., Tokyo (JP)

(21) Appl. No.: 18/993,526

(22) PCT Filed: Aug. 2, 2023
(86) PCT No.: PCT/JP2023/028349

§ 371 Date: Jan. 10, 2025

(30) Foreign Application Priority Data
Aug. 3, 2022 (JP) 2022-123766

(51) Int. Cl. H10K 85/60; C07D 403/10; H10K 50/16

(52) U.S. Cl. CPC H10K 85/6572; C07D 403/10; H10K 85/615; H10K 85/654; H10K 50/166

(57) ABSTRACT

A compound represented by the following formula (101):

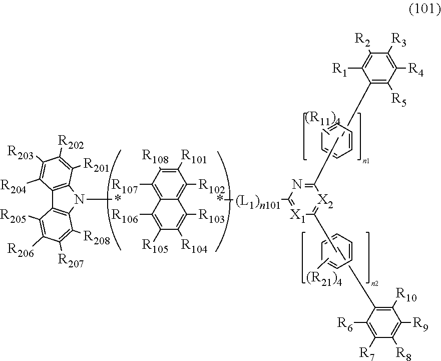

### TECHNICAL FIELD

The invention relates to a novel compound, a material for an organic electroluminescence device, an organic electroluminescence device, and an electronic apparatus.

### BACKGROUND ART

When a voltage is applied to an organic electroluminescence device (hereinafter, also referred to as an organic EL device), holes and electrons are injected into an emitting layer from an anode and cathode, respectively. Then, thus injected holes and electrons are recombined in the emitting layer, and excitons are formed therein.

Conventional organic EL devices have not yet achieved satisfactory device performance. In order to increase the device performance, materials used for organic EL devices have been gradually improved, but further enhancement of the performance is required.

Patent Documents 1 to 3 disclose use of compounds having a specific structure in an organic EL device.

### RELATED ART DOCUMENTS

### Patent Documents

[Patent Document 1] JP 2009-21336 A
[Patent Document 2] WO 2005/112519 A1
[Patent Document 3] WO 2021/157636 A1

### SUMMARY OF THE INVENTION

It is an object of the invention is to provide a high-performance organic EL device and a compound that can realize such an organic EL device.

As a result of intensive studies to achieve the above-described object, the inventors have found that a compound having a specific structure contributes to enhancement of the performance of organic EL devices, and have accomplished the invention.

According to the invention, the following compound and so on are provided.
1. A compound represented by the following formula (101):

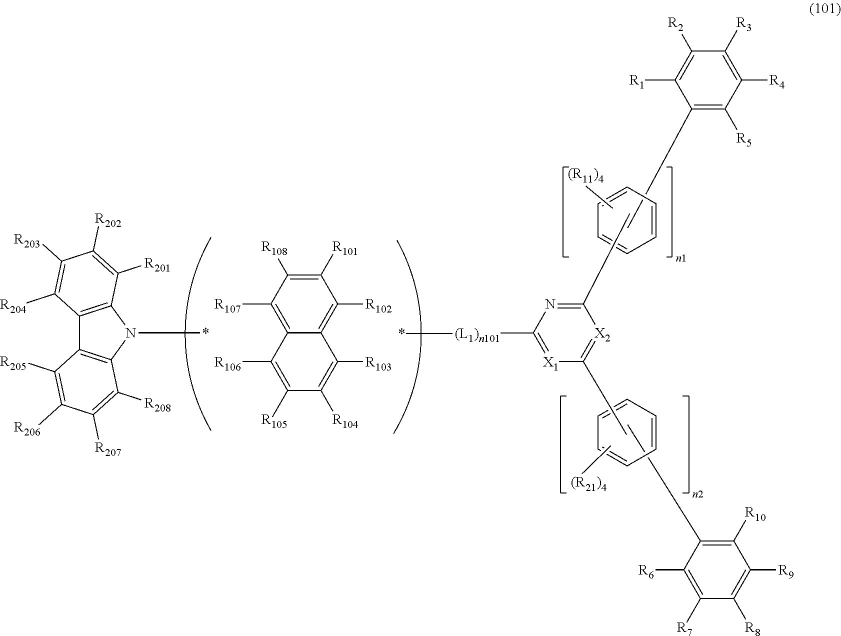

wherein in the formula (101),
one of X₁ and X₂ is N and the other is CH;
R₁ to R₁₀ are independently a hydrogen atom or a substituent R;
sets of adjacent two or more of R¹ to R₅ do not bond with each other;
sets of adjacent two or more of R₆ to R₁₀ do not bond with each other;
n1 is an integer of 0 to 3;
when n1 is 0, the structure in parentheses with n1 is a single bond;
when n1 is 2 or 3, the structures in parentheses with n1 may be the same as or different from each other;
R₁₁ is a hydrogen atom or a substituent R;
four R₁₁'s may be the same as or different from each other;
sets of adjacent two or more of four R₁₁'s do not bond with each other;
n2 is an integer of 0 to 3;
when n2 is 0, the structure in parentheses with n2 is a single bond;
when n2 is 2 or 3, the structures in parentheses with n2 may be the same as or different from each other;
R₂₁ is a hydrogen atom or a substituent R;
four R₂₁'s may be the same as or different from each other;
sets of adjacent two or more of four R₂₁'s do not bond with each other;
n101 is 0 or 1;
when n101 is 0, the structure in parentheses regarding n101 is a single bond;
L₁ is a substituted or unsubstituted phenylene group, or a substituted or unsubstituted naphthylene group;
one of R₁₀₁ to R₁₀₈ represents a single bond with (L₁) n101;
one of R₁₀₁ to R₁₀₈ which does not represent the single bond with (L₁) n101 represents a single bond with the nitrogen atom of the carbazole structure;
R₁₀₁ to R₁₀₈ which do not represent the single bonds with (L₁) n101 and with the nitrogen atom of the carbazole structure are independently a hydrogen atom or a substituent R;
sets of adjacent two or more of R₁₀₁ to R₁₀₈ do not bond with each other;
one or more sets of adjacent two or more of R₂₀₁ to R₂₀₈ form a substituted or unsubstituted aromatic hydrocarbon ring by bonding with each other, or do not bond with each other;
R₂₀₁ to R₂₀₈ which do not bond with each other are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;
   when two or more substituent R's are present, the two or more substituent R's may be the same as or different from each other;
   R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
   when two or more of each of R₉₀₁ to R₉₀₇ are present, the two or more of each of R₉₀₁ to R₉₀₇ may be the same as or different from each other.
2. The compound according to 1, wherein the compound represented by the formula (101) is a compound represented by the following formula (1):

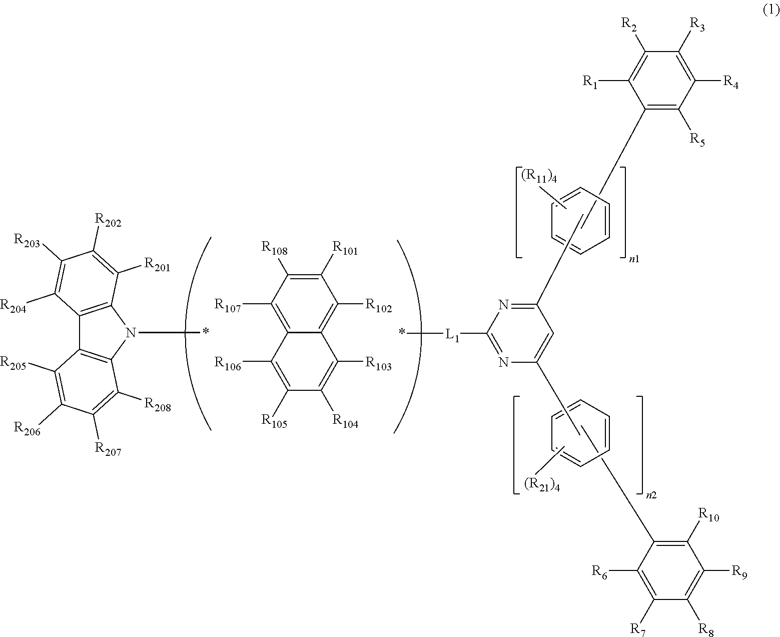

wherein in the formula (1),
R₁ to R₁₀ are independently a hydrogen atom or a substituent R;
sets of adjacent two or more of R¹ to R₅ do not bond with each other;
sets of adjacent two or more of R₆ to R₁₀ do not bond with each other;
n1 is an integer of 0 to 3;
when n1 is 0, the structure in parentheses with n1 is a single bond;
when n1 is 2 or 3, the structures in parentheses with n1 may be the same as or different from each other;
R₁₁ is a hydrogen atom or a substituent R;
four R₁₁'s may be the same as or different from each other;
sets of adjacent two or more of four R₁₁'s do not bond with each other;
n2 is an integer of 0 to 3;
when n2 is 0, the structure in parentheses with n2 is a single bond;
when n2 is 2 or 3, the structures in parentheses with n2 may be the same as or different from each other;
R₂₁ is a hydrogen atom or a substituent R;
four R₂₁'s may be the same as or different from each other;
sets of adjacent two or more of four R₂₁'s do not bond with each other;
L₁ is a substituted or unsubstituted phenylene group, or a substituted or unsubstituted naphthylene group;
one of R₁₀₁ to R₁₀₈ represents a single bond with L₁;
one of R₁₀₁ to R₁₀₈ which does not represent the single bond with L₁ represents a single bond with the nitrogen atom of the carbazole structure;
R₁₀₁ to R₁₀₈ which do not represent the single bonds with L₁ and with the nitrogen atom of the carbazole structure are independently a hydrogen atom or a substituent R;
sets of adjacent two or more of R₁₀₁ to R₁₀₈ do not bond with each other;
one or more sets of adjacent two or more of R₂₀₁ to R₂₀₈ form a substituted or unsubstituted aromatic hydrocarbon ring by bonding with each other, or do not bond with each other;
R₂₀₁ to R₂₀₈ which do not bond with each other are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;
   when two or more substituent R's are present, the two or more substituent R's may be the same as or different from each other;
   R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
   when two or more of each of R₉₀₁ to R₉₀₇ are present, the two or more of each of R₉₀₁ to R₉₀₇ may be the same as or different from each other.
3. The compound according to claim 1, wherein the compound represented by the formula (101) is a compound represented by the following formula (2):

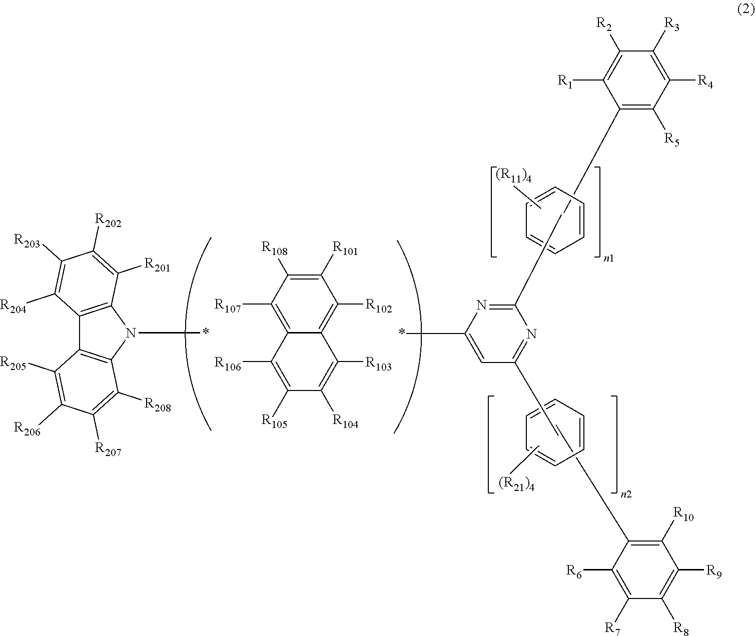

wherein in the formula (2),
R₁ to R₁₀ are independently a hydrogen atom or a substituent R;
sets of adjacent two or more of R¹ to R₅ do not bond with each other;
sets of adjacent two or more of R₆ to R₁₀ do not bond with each other;
n1 is an integer of 0 to 3;
when n1 is 0, the structure in parentheses with n1 is a single bond;
when n1 is 2 or 3, the structures in parentheses with n1 may be the same as or different from each other;
R₁₁ is a hydrogen atom or a substituent R;
four R₁₁'s may be the same as or different from each other;
sets of adjacent two or more of four R₁₁'s do not bond with each other;
a pair of R¹ and R₁₁ do not bond with each other;
a pair of R₅ and R₁₁ do not bond with each other;
n2 is an integer of 1 to 3;
when n2 is 2 or 3, the structures in parentheses with n2 may be the same as or different from each other;
R₂₁ is a hydrogen atom or a substituent R;
four R₂₁'s may be the same as or different from each other;
sets of adjacent two or more of four R₂₁'s do not bond with each other;
a pair of R₆ and R₂₁ do not bond with each other;
a pair of R₁₀ and R₂₁ do not bond with each other;
one of R₁₀₁ to R₁₀₈ represents a single bond with the carbon atom of the pyrimidine structure;
one of R₁₀₁ to R₁₀₈ which does not represent a single bond with the carbon atom of the pyrimidine structure represents a single bond with the nitrogen atom of the carbazole structure;
R₁₀₁ to R₁₀₈ which do not represent the single bonds with the carbon atom of the pyrimidine structure and with the nitrogen atom of the carbazole structure are independently a hydrogen atom or a substituent R;
sets of adjacent two or more of R₁₀₁ to R₁₀₈ do not bond with each other;
one or more sets of adjacent two or more of R₂₀₁ to R₂₀₈ form a substituted or unsubstituted aromatic hydrocarbon ring by bonding with each other, or do not bond with each other;
R₂₀₁ to R₂₀₈ which do not bond with each other are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;
   when two or more substituent R's are present, the two or more substituent R's may be the same as or different from each other;
   R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
   when two or more of each of R₉₀₁ to R₉₀₇ are present, the two or more of each of R₉₀₁ to R₉₀₇ may be the same as or different from each other.

According to the invention, a high-performance organic EL device and a compound that can realize such an organic EL device con be provided.

### BRIEF DESCRIPTION OF THE DRAWINGS

FIG. 1 is a diagram showing a schematic configuration of the organic EL device according to one aspect of the invention.

### MODE FOR CARRYING OUT THE INVENTION

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly)(for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group” is a “ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

### “Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.

### Unsubstituted Aryl Group (Specific Example Group G1A)

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
a monovalent aryl group derived by removing one hydrogen atom from the ring structures represented by any of the following general formulas (TEMP-1) to (TEMP-15).

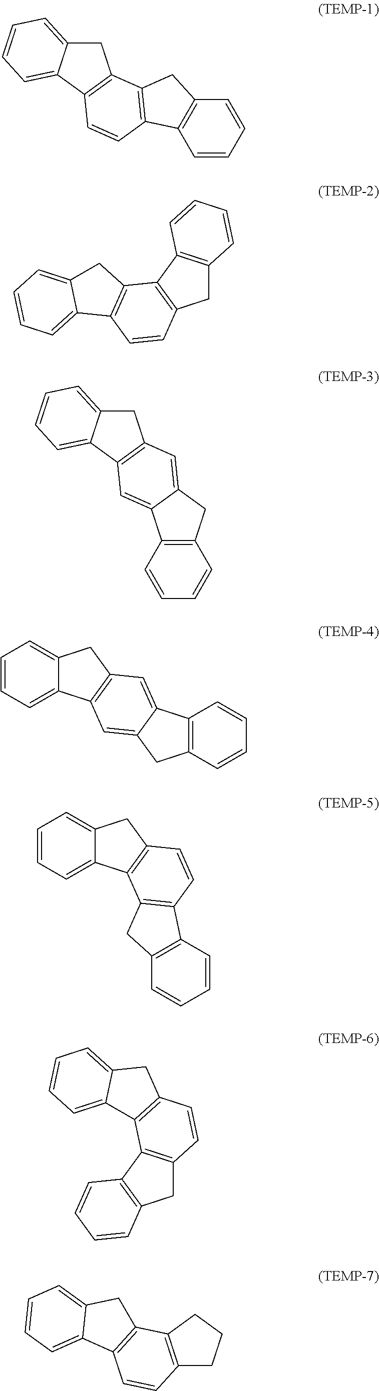

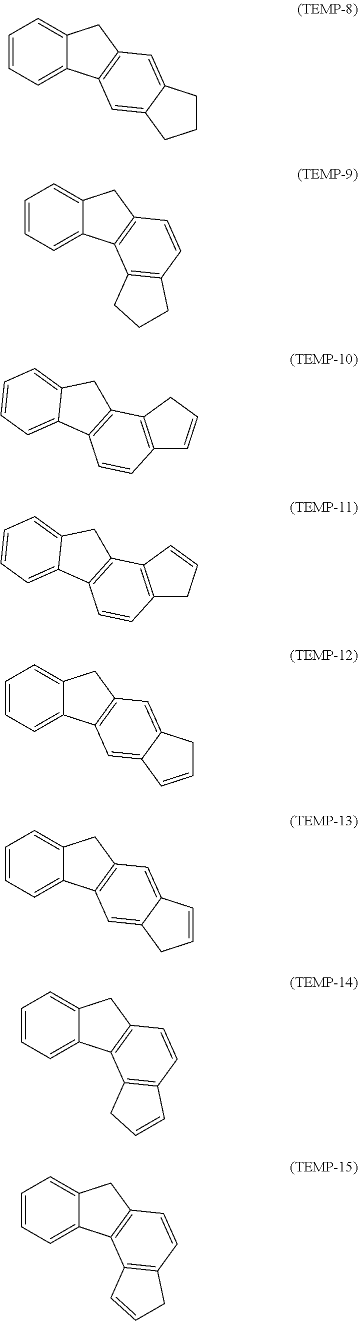

### Substituted Aryl Group (Specific Example Group G1B)

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group,
a 9,9-bis(4-methylphenyl) fluorenyl group,
a 9,9-bis(4-isopropylphenyl) fluorenyl group,
a 9,9-bis(4-t-butylphenyl) fluorenyl group,
a cyanophenyl group,
a triphenylsilylphenyl group,
a trimethylsilylphenyl group,
a phenylnaphthyl group,
a naphthylphenyl group, and
a group in which one or more hydrogen atoms of a monovalent group derived from the ring structures represented by any of the general formulas (TEMP-1) to (TEMP-15) are substituted by a substituent.

### “Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group” is a “heterocyclic group unsubstituted by a substituent”, and the substituted heterocyclic group refers to the case where the “substituted or unsubstituted heterocyclic group” is a “heterocyclic group substituted by a substituent”). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by any of the following general formulas (TEMP-16) to (TEMP-33)(specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by any of the following general formulas (TEMP-16) to (TEMP-33) are substituted by a substituent (specific example group G2B4).

### Unsubstituted Heterocyclic Group Containing a Nitrogen Atom (Specific Example Group G2A1)

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

### Unsubstituted Heterocyclic Group Containing an Oxygen Atom (Specific Example Group G2A2)

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### Unsubstituted Heterocyclic Group Containing a Sulfur Atom (Specific Example Group G2A3)

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).
Monovalent heterocyclic group derived by removing one hydrogen atom from the ring structures represented by any of the following general formulas (TEMP-16) to (TEMP-33)(specific example group G2A4):

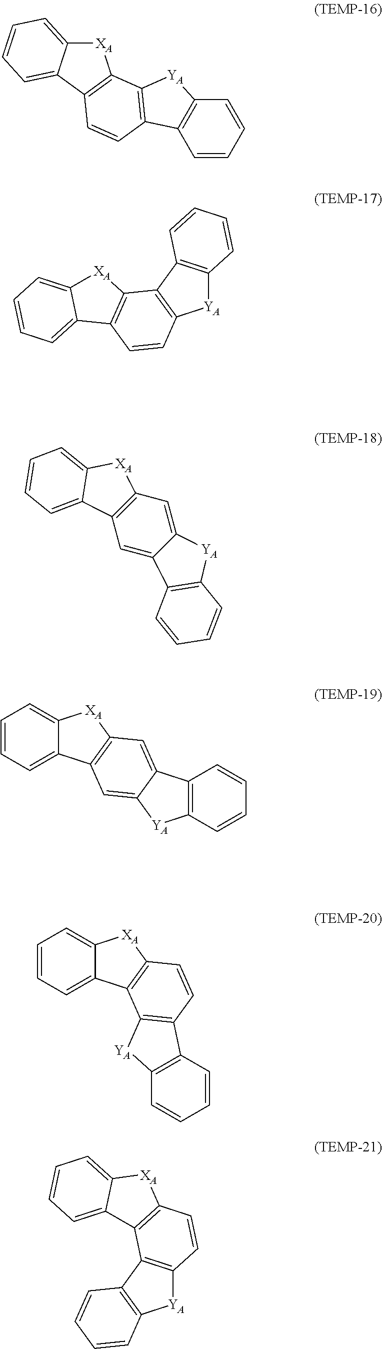

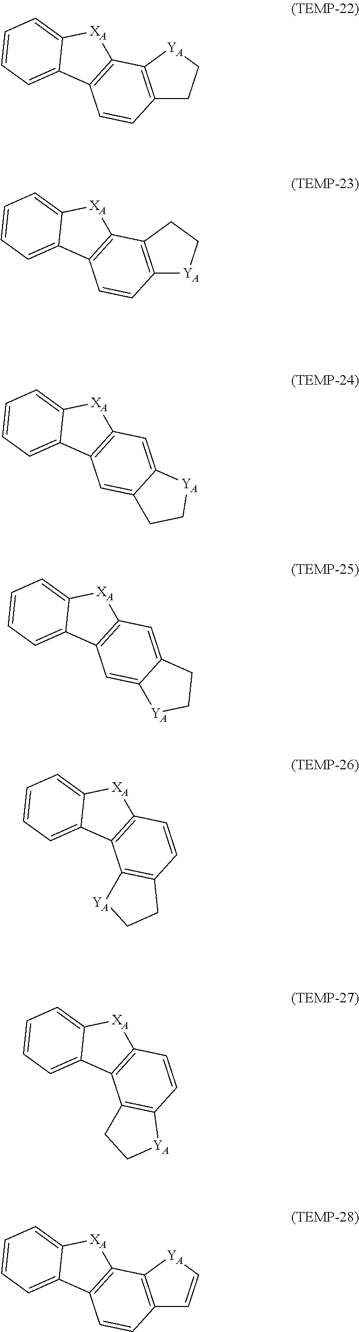

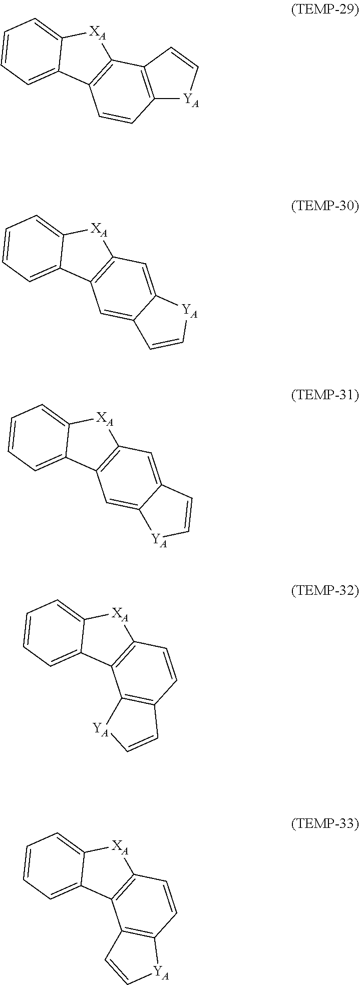

In the general formulas (TEMP-16) to (TEMP-33), X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by any of the general formulas (TEMP-16) to (TEMP-33) includes a monovalent group derived by removing one hydrogen atom from these NH or CH₂.

### Substituted Heterocyclic Group Containing a Nitrogen Atom (Specific Example Group G2B1)

a (9-phenyl) carbazolyl group,
a (9-biphenylyl) carbazolyl group,
a (9-phenyl)phenylcarbazolyl group,
a (9-naphthyl) carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

### Substituted Heterocyclic Group Containing an Oxygen Atom (Specific Example Group G2B2)

a phenyldibenzofuranyl group,
a methyldibenzofuranyl group,
a t-butyldibenzofuranyl group, and
a monovalent residue of spiro[9H-xanthene-9,9′-[9H]fluorene].

### Substituted Heterocyclic Group Containing a Sulfur Atom (Specific Example Group G2B3)

a phenyldibenzothiophenyl group,
a methyldibenzothiophenyl group,
a t-butyldibenzothiophenyl group, and
a monovalent residue of spiro[9H-thioxanthene-9,9′-[9H]fluorene].
Group in which one or more hydrogen atoms of the monovalent heterocyclic group derived from the ring structures represented by any of the following general formulas (TEMP-16) to (TEMP-33) are substituted by a substituent (specific example group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.

### “Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an “alkyl group substituted by a substituent”). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

### Unsubstituted Alkyl Group (Specific Example Group G3A)

a methyl group,
an ethyl group,
a n-propyl group,
an isopropyl group,
a n-butyl group,
an isobutyl group,
a s-butyl group, and
a t-butyl group.

### Substituted Alkyl Group (Specific Example Group G3B)

a heptafluoropropyl group (including isomers),
a pentafluoroethyl group,
a 2,2,2-trifluoroethyl group, and
a trifluoromethyl group.

### “Substituted or Unsubstituted Alkenyl Group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group” is a “alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

### Unsubstituted Alkenyl Group (Specific Example Group G4A)

a vinyl group,
an allyl group,
a 1-butenyl group,
a 2-butenyl group, and
a 3-butenyl group.

### Substituted Alkenyl Group (Specific Example Group G4B)

a 1,3-butanedienyl group,
a 1-methylvinyl group,
a 1-methylallyl group,
a 1,1-dimethylallyl group,
a 2-methylally group, and
a 1,2-dimethylallyl group.

### “Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

### Unsubstituted Alkynyl Group (Specific Example Group G5A)

an ethynyl group.

### “Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group” is a “cycloalkyl group unsubstituted by a substituent”, and the substituted cycloalkyl group refers to the case where the “substituted or unsubstituted cycloalkyl group” is a “cycloalkyl group substituted by a substituent”). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

### Unsubstituted Cycloalkyl Group (Specific Example Group G6A)

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Specific Example Group G6B)

a 4-methylcyclohexyl group.
   “Group Represented by —Si(R₉₀₁)(R₉₀₂)(R₉₀₃)”

Specific examples of the group represented by —Si(R₉₀₁)(R₉₀₂)(R₉₀₃) described in this specification (specific example group G7) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —Si(G1)(G1)(G1) are the same or different.

Plural G2's in —Si(G1)(G2)(G2) are the same or different.

Plural G1's in —Si(G1)(G1)(G2) are the same or different.

Plural G2's in —Si(G2)(G2)(G2) are be the same or different.

Plural G3's in —Si(G3)(G3)(G3) are the same or different.

Plural G6's in —Si(G6)(G6)(G6) are be the same or different.

### “Group Represented by —O—(R₉₀₄)”

Specific examples of the group represented by —O—(R₉₀₄) in this specification (specific example group G8) include:
—O(G1),
—O(G2),
—O(G3), and
—O(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

### “Group Represented by —S—(R₉₀₅)”

Specific examples of the group represented by —S—(R₉₀₅) in this specification (specific example group G9) include:
—S(G1),
—S(G2),
—S(G3), and
—S(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group Represented by —N(R₉₀₆)(R₉₀₇)”

Specific examples of the group represented by —N(R₉₀₆)(R₉₀₇) in this specification (specific example group G10) include:
—N(G1)(G1),
—N(G2)(G2),
—N(G1)(G2),
—N(G3)(G3), and
—N(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in-N(G1)(G1) are the same or different.

Plural G2's in-N(G2)(G2) are the same or different.

Plural G3's in-N(G3)(G3) are the same or different.

Plural G6's in-N(G6)(G6) are the same or different.

### “Halogen Atom”

Specific examples of the “halogen atom” described in this specification (specific example group G11) include a fluorine atom, a chlorine atom, a bromine atom, an iodine atom, and the like.

### “Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

### “Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

### “Substituted or Unsubstituted Alkoxy Group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

### “Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

### “Substituted or Unsubstituted Aryloxy Group”

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

### “Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

### “Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.
“Substituted or unsubstituted aralkyl group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an a-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl) carbazolyl group (a (9-phenyl) carbazol-1-yl group, a (9-phenyl) carbazol-2-yl group, a (9-phenyl) carbazol-3-yl group, or a (9-phenyl) carbazol-4-yl group), a (9-biphenylyl) carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically any of the following groups, unless otherwise specified in this specification.

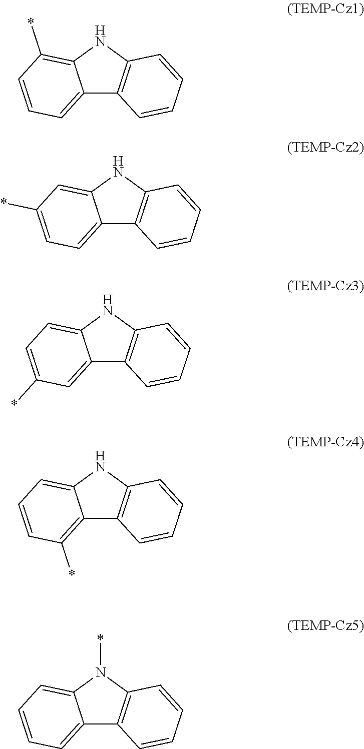 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In this specification, the (9-phenyl) carbazolyl group is specifically any of the following groups, unless otherwise specified in this specification.

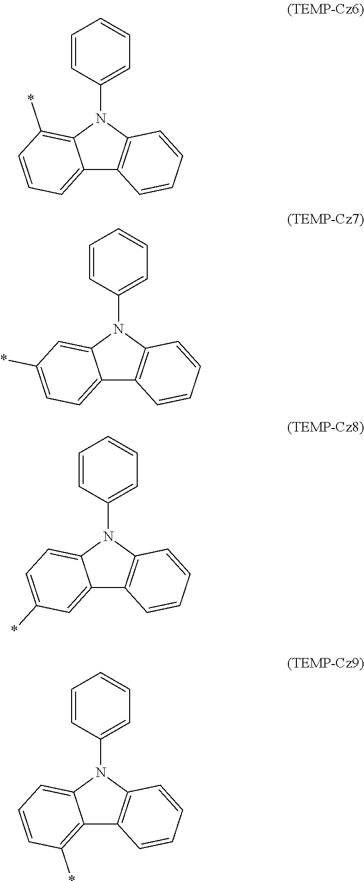

In the general formulas (TEMP-Cz1) to (TEMP-Cz9), * represents a bonding site.

In this specification, the dibenzofuranyl group and the dibenzothiophenyl group are specifically any of the following groups, unless otherwise specified in this specification.

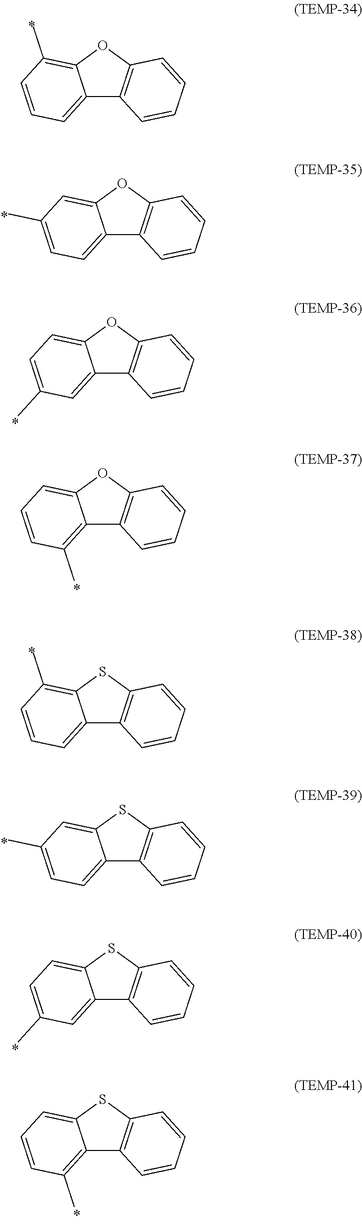

In the general formulas (TEMP-34) to (TEMP-41), * represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

### “Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

### “Substituted or Unsubstituted Divalent Heterocyclic Group”

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.

### “Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

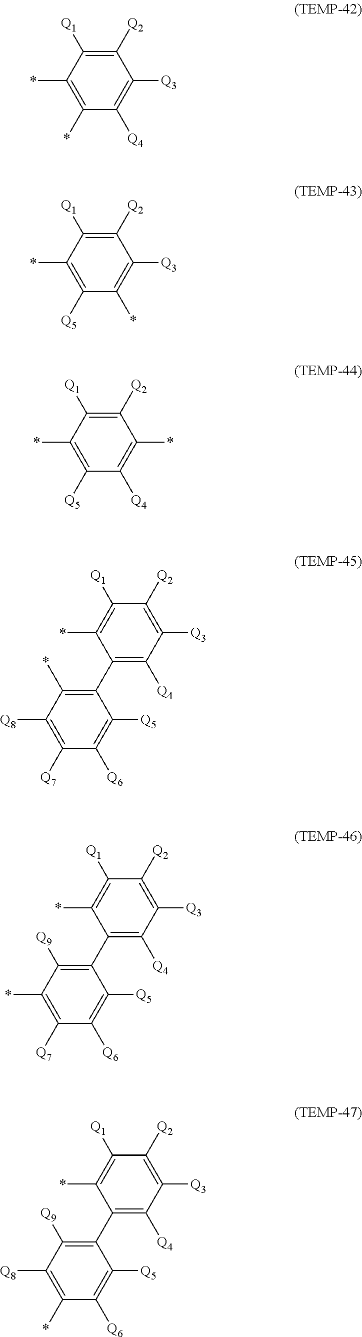

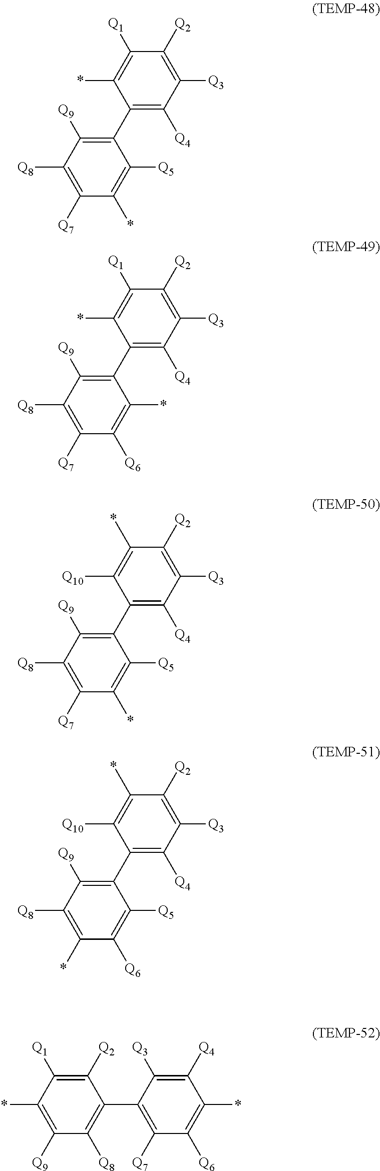

In the general formulas (TEMP-42) to (TEMP-52), Q₁ to Q₁₀ are independently a hydrogen atom or a substituent.

In the general formulas (TEMP-42) to (TEMP-52), * represents a bonding site.

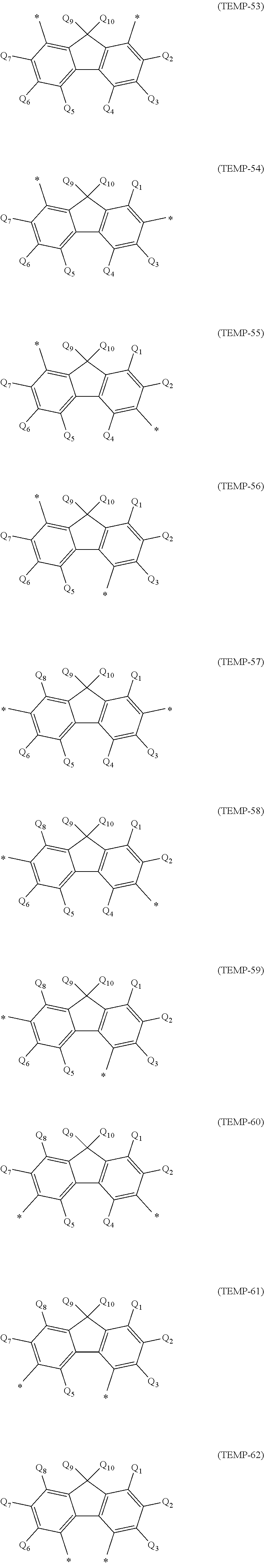

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding site.

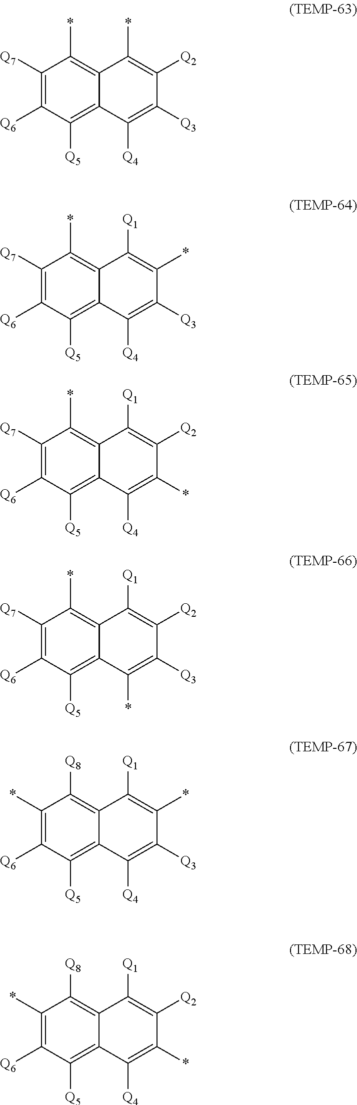

In the general formulas (TEMP-63) to (TEMP-68), Q₁ to Qs are independently a hydrogen atom or a substituent.

In the general formulas (TEMP-63) to (TEMP-68), * represents a bonding site.

The substituted or unsubstituted divalent heterocyclic group described in this specification is preferably any group of the following general formulas (TEMP-69) to (TEMP-102), unless otherwise specified in this specification.

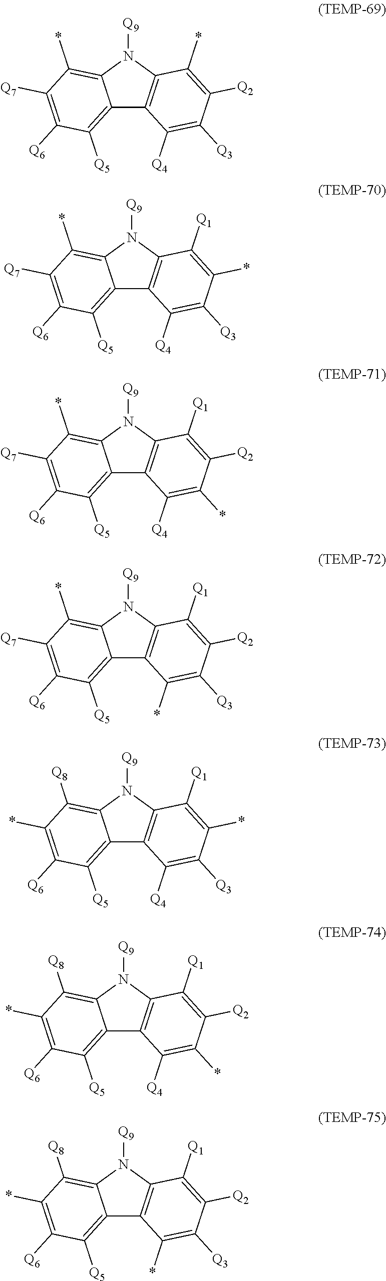

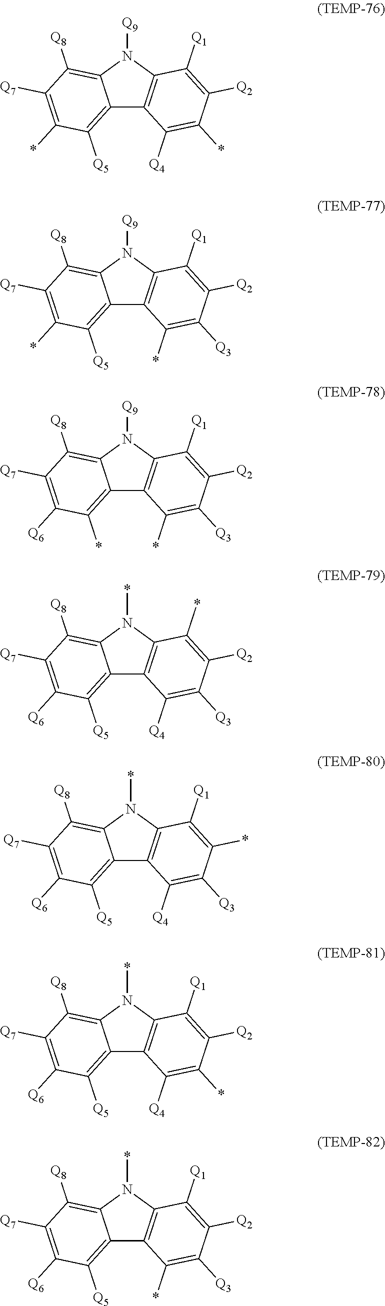

In the general formulas (TEMP-69) to (TEMP-82), Q₁ to Q₉ are independently a hydrogen atom or a substituent.

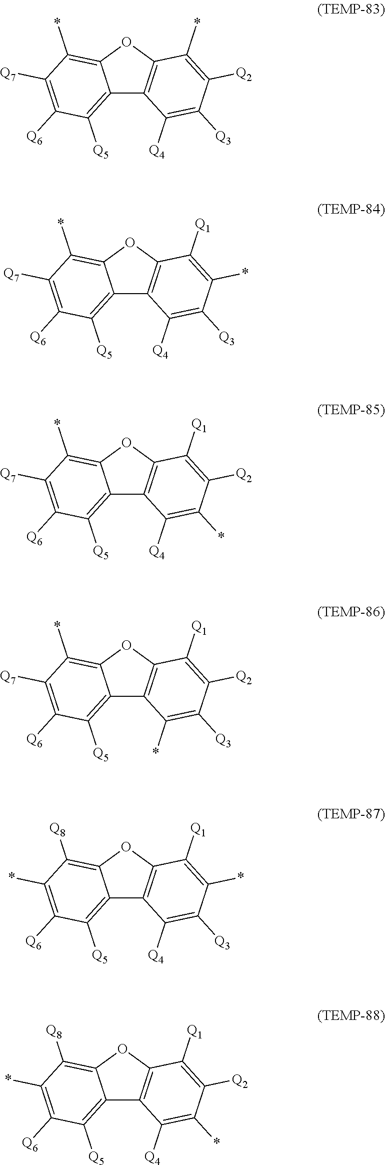

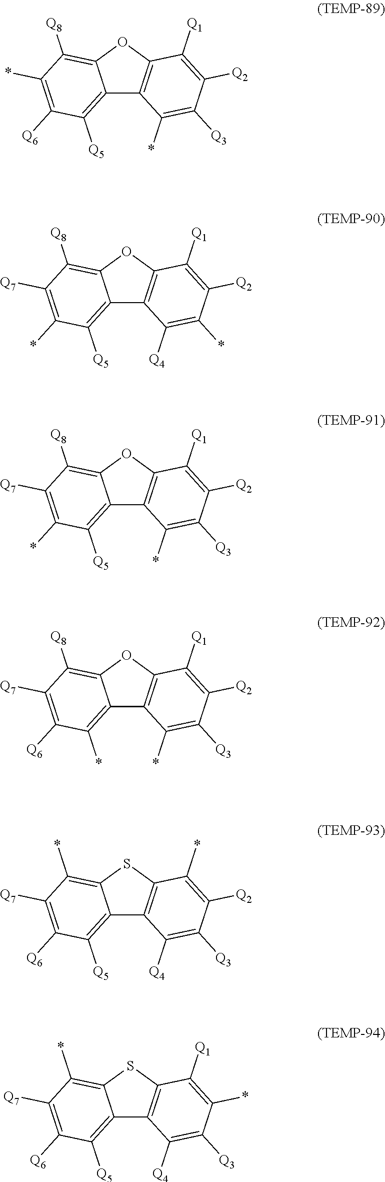

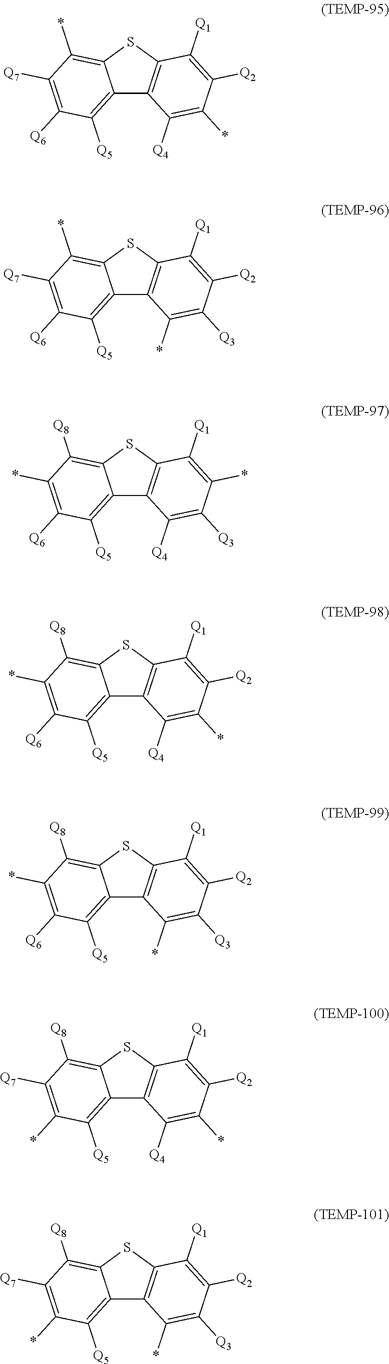

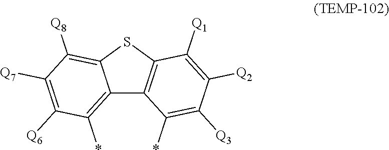

In the general formulas (TEMP-83) to (TEMP-102), Q₁ to Q₈ are independently a hydrogen atom or a substituent.

The above is the explanation of the “Substituent described in this specification.”

“the Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

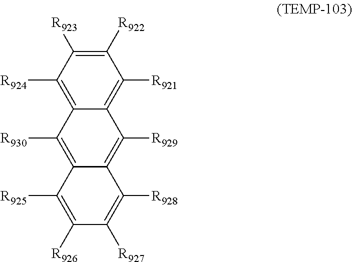

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring QB by bonding with each other, the anthracene compound represented by the general formula (TEMP-103) is represented by the following general formula (TEMP-104).

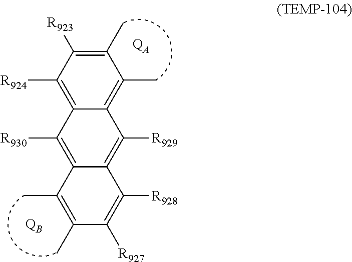

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Q_{C} by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Q_{C} share R₉₂₂.

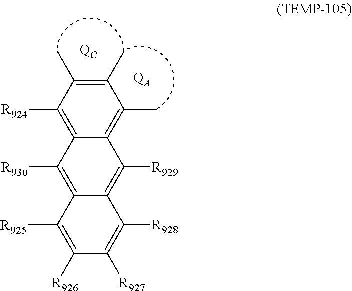

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring QB formed in the general formula (TEMP-104) are independently a “monocycle” or a “fused ring.” The ring Q_{A} and the ring Q_{C} formed in the general formula (TEMP-105) are “fused ring.” The ring Q_{A} and ring Q_{C} of the general formula (TEMP-105) are fused ring by fusing the ring Q_{A} and the ring Q_{C} together. When the ring Q_{A} of the general formula (TMEP-104) is a benzene ring, the ring Q_{A} is a monocycle. When the ring Q_{A} of the general formula (TMEP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and/or triple bonds in the ring structure (e.g., cyclohexene, cyclohexadiene, etc.), and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other” (the case where “forming a ring by bonding with each other”).

### Substituent in the Case of “Substituted or Unsubstituted”

In one embodiment in this specification, the substituent (in this specification, sometimes referred to as an “arbitrary substituent”) in the case of “substituted or unsubstituted” is, for example, a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, and
an unsubstituted heterocyclic group including 5 to 50 ring atoms,
wherein, R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted heterocyclic group including 5 to 50 ring atoms.

When two or more R₉₀₁'s are present, the two or more R₉₀₁'s may be the same or different.

When two or more R₉₀₂'s are present, the two or more R₉₀₂'s may be the same or different.

When two or more R₉₀₃'s are present, the two or more R₉₀₃'s may be the same or different.

When two or more R₉₀₄'s are present, the two or more R₉₀₄'s may be the same or different.

When two or more R₉₀₅'s are present, the two or more R₉₀₅'s may be the same or different.

When two or more R₉₀₆'s are present, the two or more R₉₀₆'s may be the same or different.

When two or more R₉₀₇'s are present, the two or more R₉₀₇'s may be the same or different.

In one embodiment, the substituent in the case of “substituted or unsubstituted” is a group selected from the group consisting of:
an alkyl group including 1 to 50 carbon atoms,
an aryl group including 6 to 50 ring carbon atoms, and
a heterocyclic group including 5 to 50 ring atoms.

In one embodiment, the substituent in the case of “substituted or unsubstituted” is a group selected from the group consisting of:
an alkyl group including 1 to 18 carbon atoms,
an aryl group including 6 to 18 ring carbon atoms, and
a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [Novel Compound]

A compound according to one aspect of the invention is represented by the following formula (101):

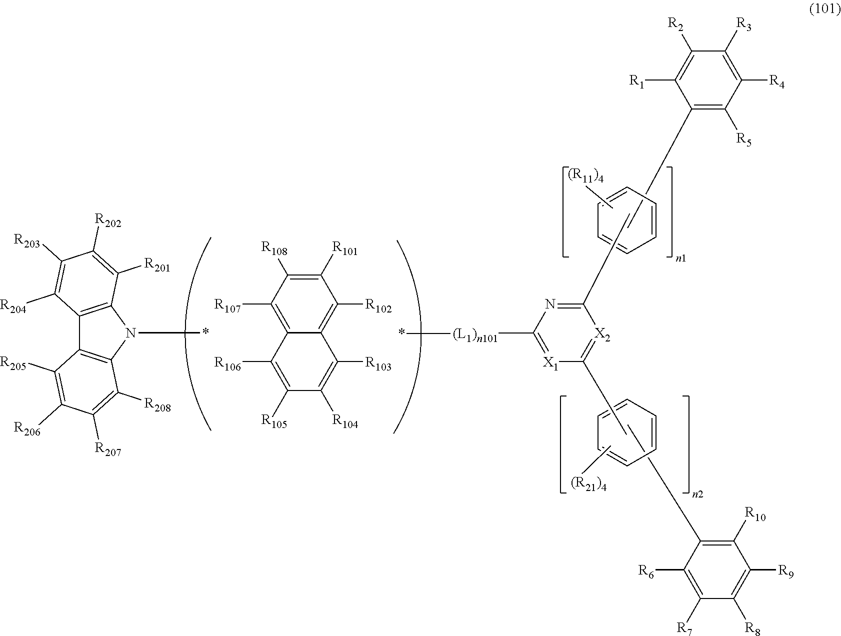

wherein in the formula (101),
one of X₁ and X₂ is N and the other is CH;
R₁ to R₁₀ are independently a hydrogen atom or a substituent R;
sets of adjacent two or more of R¹ to R₅ do not bond with each other;
sets of adjacent two or more of R₆ to R₁₀ do not bond with each other;
n1 is an integer of 0 to 3;
when n1 is 0, the structure in parentheses with n1 is a single bond;
when n1 is 2 or 3, the structures in parentheses with n1 may be the same as or different from each other;
R₁₁ is a hydrogen atom or a substituent R;
four R₁₁'s may be the same as or different from each other;
sets of adjacent two or more of four R₁₁'s do not bond with each other;
n2 is an integer of 0 to 3;
when n2 is 0, the structure in parentheses with n2 is a single bond;
when n2 is 2 or 3, the structures in parentheses with n2 may be the same as or different from each other;
R₂₁ is a hydrogen atom or a substituent R;
four R₂₁'s may be the same as or different from each other;
sets of adjacent two or more of four R₂₁'s do not bond with each other;
n101 is 0 or 1;
when n101 is 0, the structure in parentheses with n101 is a single bond; L₁ is a substituted or unsubstituted phenylene group, or a substituted or unsubstituted naphthylene group;
one of R₁₀₁ to R₁₀₈ represents a single bond with (L₁) n101;
one of R₁₀₁ to R₁₀₈ which does not represent a single bond with (L₁) n101 represents a single bond with the nitrogen atom of the carbazole structure;
R₁₀₁ to R₁₀₈ which do not represent the single bonds with (L₁) n101 and with the nitrogen atom of the carbazole structure are independently a hydrogen atom or a substituent R;
sets of adjacent two or more of R₁₀₁ to R₁₀₈ do not bond with each other;
one or more sets of adjacent two or more of R₂₀₁ to R₂₀₈ form a substituted or unsubstituted aromatic hydrocarbon ring by bonding with each other, or do not bond with each other;
R₂₀₁ to R₂₀₈ which do not bond with each other are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;
when two or more substituent R's are present, the two or more substituent R's may be the same as or different from each other;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ may be the same as or different from each other.

The structure in parentheses regarding n1 in the formula (101), that is, the structure represented by the following formula (111) will be described.

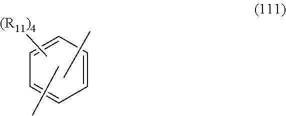

In the structure represented by the formula (111), two of six carbon atoms constituting the benzene ring are bonded with neighboring structures via a single bond, respectively, and the rest four carbon atoms are bonded with R₁₁, respectively.

In other words, the structure represented by the formula (111) may become any of three structures represented by the formulas (111a) to (111c):

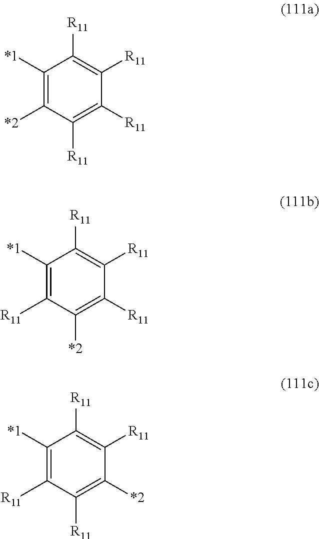

In the formulas (111a) to (111c), *1 and *2 respectively represent a single bond with neighboring structures. R₁₁ is as defined in the formula (101).

For example, when n1 is 3, and the three structures represented by the formula (111) are linked, in order from the pyrimidine structure side in the formula (101), the structure represented by the formula (111c), the structure represented by the formula (111b), and the structure represented by the formula (111a), the overall structure thereof is as follows:

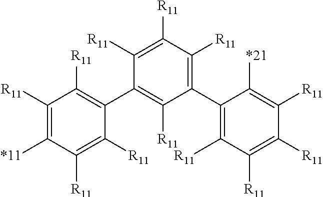

In the above formula, *11 represents a single bond with a pyrimidine structure; *21 represents a single bond with a phenyl group; and R₁₁ is as defined in the formula (101).

As is obvious from the definitions, in the formula (101), when n1 is 0, the structure represented by the formula (111) is not present, and the pyrimidine structure and the phenyl group are directly bonded via a single bond.

The same applies to the structure in parentheses with n2 in the formula (101), that is, the structure represented by the following formula (211):

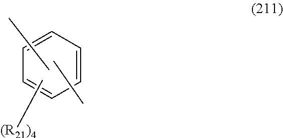

Specifically, in the structure represented by the formula (211), two of six carbon atoms constituting the benzene ring are bonded with neighboring structures via a single bond, respectively, and the rest four carbon atoms are bonded with R₂₁, respectively.

In other words, the structure represented by the formula (211) may become any of three structures represented by the formulas (211a) to (211c).

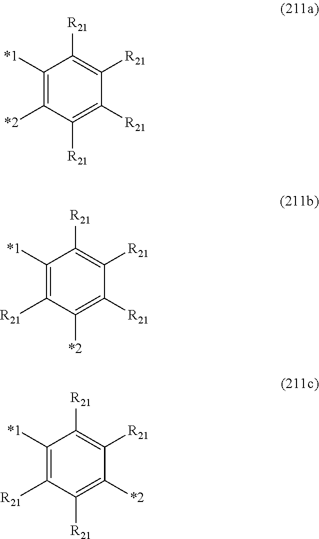

In the formulas (211a) to (211c), *1 and *2 respectively represent a single bond with neighboring structures. R₂₁ are as defined in the formula (101).

As is obvious from the definitions, in the formula (101), when n2 is 0, the structure represented by the formula (211) is not present, and the pyrimidine structure and the phenyl group are directly bonded via a single bond.
when n101 is 0, the structure in parentheses with n101 is a single bond.

As is obvious from the definitions, when n101 is 0, the pyrimidine structure and the naphthalene structure are directly bonded via a single bond.

In one embodiment, X₁ is N and X₂ is CH.

In one embodiment, X₁ is CH and X₂ is N.

In one embodiment, R₁₁ is a hydrogen atom.

In one embodiment, R₂₁ is a hydrogen atom.

In one embodiment, n1 and n2 are independently 0 or 1.

In one embodiment, n1 and n2 are 0.

In one embodiment, n1 is 0 and n2 is 1.

In one embodiment, R₁ to R₁₀ are hydrogen atoms.

In one embodiment, L₁ is a substituted or unsubstituted phenylene group.

In one embodiment, R₁₀₂ represents a single bond with L₁.

In one embodiment, R₁₀₇ represents a single bond with the nitrogen atom of the carbazole structure.

In one embodiment, R₁₀₁ to R₁₀₈ which do not represent a single bond are hydrogen atoms.

As is obvious from the definitions, one of R₁₀₁ to R₁₀₈ and one of R₂₀₁ to R₂₀₈ do not bond with each other. That is, in the formula (101), no ring is not formed between the naphthalene skeleton and the carbazole skeleton.

In one embodiment, one or more sets of adjacent two or more of R₂₀₁ to R₂₀₈ do not bond with each other.

In one embodiment, R₂₀₁ to R₂₀₈ are hydrogen atoms.

In one embodiment, the compound represented by the formula (101) is a compound represented by the following formula (1):

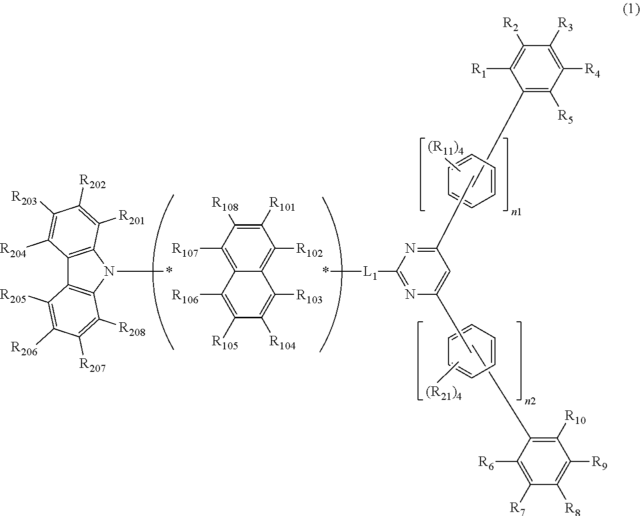

wherein in the formula (1),
R₁ to R₁₀ are independently a hydrogen atom or a substituent R;
sets of adjacent two or more of R¹ to R₅ do not bond with each other;
sets of adjacent two or more of R₆ to R₁₀ do not bond with each other;
n1 is an integer of 0 to 3;
when n1 is 0, the structure in parentheses with n1 is a single bond;
when n1 is 2 or 3, the structures in parentheses with n1 may be the same as or different from each other;
R₁₁ is a hydrogen atom or a substituent R;
four R₁₁'s may be the same as or different from each other;
sets of adjacent two or more of four R₁₁'s do not bond with each other;
n2 is an integer of 0 to 3;
when n2 is 0, the structure in parentheses with n2 is a single bond;
when n2 is 2 or 3, the structures in parentheses with n2 may be the same as or different from each other;
R₂₁ is a hydrogen atom or a substituent R;
four R₂₁'s may be the same as or different from each other;
sets of adjacent two or more of four R₂₁'s do not bond with each other;
L₁ is a substituted or unsubstituted phenylene group, or a substituted or unsubstituted naphthylene group;
one of R₁₀₁ to R₁₀₈ represents a single bond with L₁;
one of R₁₀₁ to R₁₀₈ which does not represent a single bond with L₁ represents a single bond with the nitrogen atom of the carbazole structure;
R₁₀₁ to R₁₀₈ which do not represent the single bonds with L₁ and with the nitrogen atom of the carbazole structure are independently a hydrogen atom or a substituent R;
sets of adjacent two or more of R₁₀₁ to R₁₀₈ do not bond with each other;
one or more sets of adjacent two or more of R₂₀₁ to R₂₀₈ form a substituted or unsubstituted aromatic hydrocarbon ring by bonding with each other, or do not bond with each other;
R₂₀₁ to R₂₀₈ which do not bond with each other are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;
when two or more substituent R's are present, the two or more substituent R's may be the same as or different from each other;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ may be the same as or different from each other.

In one embodiment, R₁₁ in the formula (1) is a hydrogen atom.

In one embodiment, R₂₁ in the formula (1) is a hydrogen atom.

In one embodiment, n1 and n2 in the formula (1) are 0.

In one embodiment, R₁ to R₁₀ in the formula (1) are hydrogen atoms.

In one embodiment, L₁ in the formula (1) is a substituted or unsubstituted phenylene group.

In one embodiment, R₁₀₂ in the formula (1) represents a single bond with L₁.

In one embodiment, R₁₀₇ in the formula (1) represents a single bond with the nitrogen atom of the carbazole structure.

In one embodiment, R₁₀₁ to R₁₀₈ which do not represent single bonds with L₁ and with the nitrogen atom of the carbazole structure in the formula (1) are hydrogen atoms.

As is obvious from the definitions, one of R₁₀₁ to R₁₀₈ and one of R₂₀₁ to R₂₀₈ do not bond with each other. That is, in the formula (1), no ring is formed between the naphthalene skeleton and the carbazole skeleton.

In one embodiment, one or more sets of adjacent two or more of R₂₀₁ to R₂₀₈ in the formula (1) do not bond with each other.

In one embodiment, R₂₀₁ to R₂₀₈ in the formula (1) are hydrogen atoms.

In one embodiment, the compound represented by the formula (1) is a compound represented by the following formula (11):

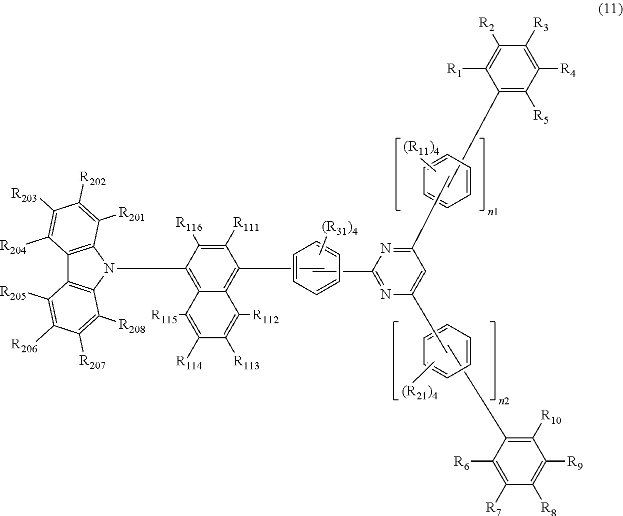

wherein in the formula (11),
R₁ to R₁₀, R₁₁, R₂₁, n1, n2, and R₂₀₁ to R₂₀₈ are as defined in the formula (1); R₃₁ is a hydrogen atom or a substituent R;
four R₃₁'s may be the same as or different from each other;
sets of adjacent two or more of four R₃₁'s do not bond with each other; R₁₁₁ to R₁₁₆ are independently a hydrogen atom or a substituent R;
sets of adjacent two or more of R₁₁₁ to R₁₁₆ do not bond with each other; and
the substituent R is as defined in the formula (1).

In the formula (11), the phenylene structure with which R₃₁ bonds, that is, a structure represented by the following formula (311) will be described:

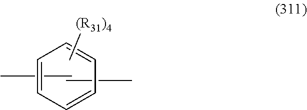

In the structure represented by the formula (311), two of six carbon atoms constituting the benzene ring are bonded with neighboring structures via a single bond, respectively, and the rest four carbon atoms are bonded with R₃₁, respectively.

In other words, the structure represented by the formula (311) may become any of three structures represented by the formula (311a) to (311c):

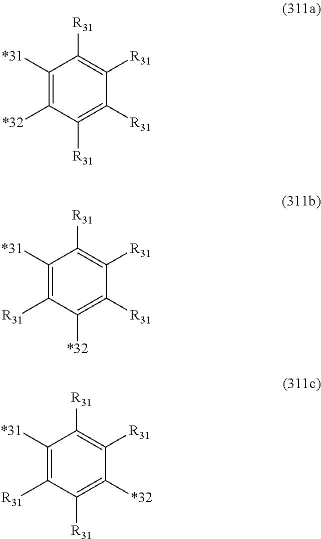

wherein in the formulas (311a) to (311c), *31 represents a single bond with the neighboring pyrimidine structure and *32 represents a single bond with the neighboring naphthalene skeleton. R₃₁ is as defined in the formula (11).

For example, when the structure represented by the formula (311) is a structure represented by the formula (311c), the compound represented by the formula (11) is represented by the following formula (11c):

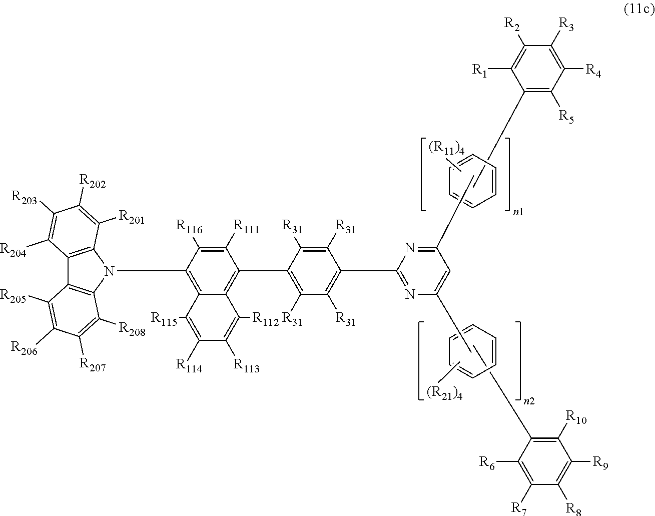

wherein in the formula (11c), R₁ to R₁₀, R₁₁, R₂₁, R₃₁, R₁₁₁ to R₁₁₆, n1, n2, and R₂₀₁ to R₂₀₈ are as defined in the formula (11).

In one embodiment, R₃₁ is a hydrogen atom.

In one embodiment, R₁₁₁ to R₁₁₆ are hydrogen atoms.

In one embodiment, the compound represented by the formula (101) is a compound represented by the following formula (2):

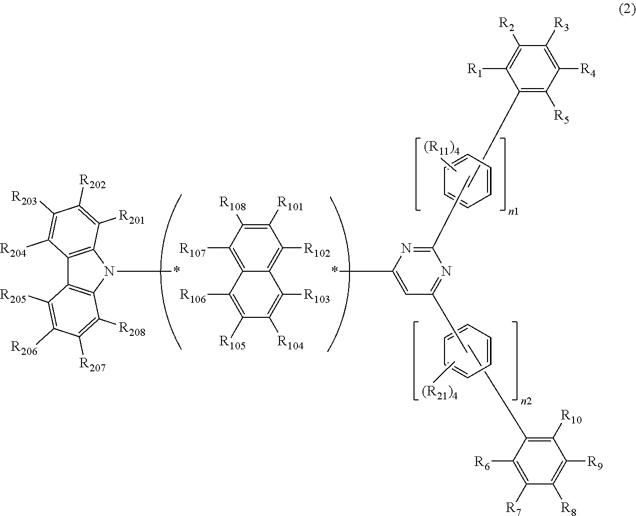

wherein in the formula (2),
R₁ to R₁₀ are independently a hydrogen atom or a substituent R;
sets of adjacent two or more of R¹ to R₅ do not bond with each other;
sets of adjacent two or more of R₆ to R₁₀ do not bond with each other;
n1 is an integer of 0 to 3;
when n1 is 0, the structure in parentheses with n1 is a single bond;
when n1 is 2 or 3, the structures in parentheses with n1 may be the same as or different from each other;
R₁₁ is a hydrogen atom or a substituent R;
four R₁₁'s may be the same as or different from each other;
sets of adjacent two or more of four R₁₁'s do not bond with each other;
n2 is an integer of 1 to 3;
when n2 is 2 or 3, the structures in parentheses with n2 may be the same as or different from each other;
R₂₁ is a hydrogen atom or a substituent R;
four R₂₁'s may be the same as or different from each other;
sets of adjacent two or more of four R₂₁'s do not bond with each other;
one of R₁₀₁ to R₁₀₈ represents a single bond with the carbon atom of the pyrimidine structure;
one of R₁₀₁ to R₁₀₈ which does not represent a single bond with the carbon atom of the pyrimidine structure represents a single bond with the nitrogen atom of the carbazole structure;
R₁₀₁ to R₁₀₈ which do not represent the single bonds with the carbon atom of the pyrimidine structure and with the nitrogen atom of the carbazole structure are independently a hydrogen atom or a substituent R;
sets of adjacent two or more of R₁₀₁ to R₁₀₈ do not bond with each other;
one or more sets of adjacent two or more of R₂₀₁ to R₂₀₈ form a substituted or unsubstituted aromatic hydrocarbon ring by bonding with each other, or do not bond with each other;
R₂₀₁ to R₂₀₈ which do not bond with each other are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;
when two or more substituent R's are present, the two or more substituent R's may be the same as or different from each other;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ may be the same as or different from each other.

In one embodiment, in the formula (2), R₁₀₂ represents a single bond with the carbon atom of the pyrimidine structure, and R₁₀₇ represents a single bond with the nitrogen atom of the carbazole structure.

In one embodiment, one or more sets of adjacent two or more of R₂₀₁ to R₂₀₈ in the formula (2) do not bond with each other.

In one embodiment, the compound represented by the formula (2) is a compound represented by the following formula (21):

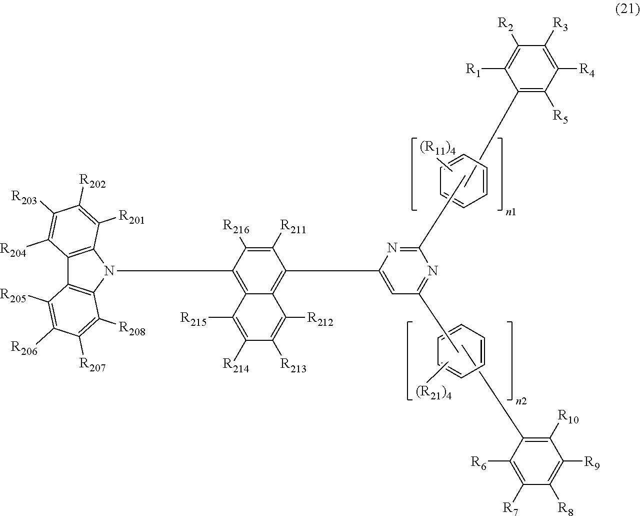

wherein in the formula (21),
R₁ to R₁₀, R₁₁, R₂₁, n1, n2, and R₂₀₁ to R₂₀₈ are as defined in the formula (2); R₂₁₁ to R₂₁₆ are independently a hydrogen atom or a substituent R;
sets of adjacent two or more of R₂₁₁ to R₂₁₆ do not bond with each other; and the substituent R is as defined in the formula (2).

In one embodiment, n1 in the formula (2) is 0.

In one embodiment, R₁ to R₁₀ in the formula (2) are hydrogen atoms.

In one embodiment, any of R₂₀₁ to R₂₀₈ in the formula (2) is a substituted or unsubstituted aryl group including 6 to 50 ring carbon atoms,
the rest of R₂₀₁ to R₂₀₈ are independently selected from the group consisting of:
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group, and
a substituted or unsubstituted monovalent heterocyclic group including 5 to 50 ring atoms.

In one embodiment, R₂₀₃ in the formula (2) is a substituted or unsubstituted aryl group including 1 to 50 ring carbon atoms.

In one embodiment, R₂₀₃ in the formula (2) is a substituted or unsubstituted phenyl group.

In one embodiment, R₂₀₁ to R₂₀₂ and R₂₀₄ to R₂₀₈ in the formula (2) are hydrogen atoms.

In one embodiment, R₂₁₁ to R₂₁₆ in the formula (2) are hydrogen atoms.

In one embodiment, the substituent in the case of “substituted or unsubstituted,” and the substituent R are a group independently selected from the group consisting of:
an alkyl group including 1 to 50 carbon atoms,
an aryl group including 6 to 50 ring carbon atoms, and
a monovalent heterocyclic group including 5 to 50 ring atoms.

In one embodiment, the substituent in the case of “substituted or unsubstituted,” and the substituent R are a group independently selected from the group consisting of:
an alkyl group including 1 to 18 carbon atoms,
an aryl group including 6 to 18 ring carbon atoms, and
a monovalent heterocyclic group including 5 to 18 ring atoms.

In one embodiment, the compound represented by the formula (101) has no deuterium atom as a hydrogen atom in the molecule.

In this specification, “having no deuterium atom as hydrogen atom” means that the proportion of deuterium atoms to the sum of protium atoms and deuterium atoms in all hydrogen atoms in the molecule is lower than or equal to the natural abundance ratio thereof. In other words, the compound according to one aspect of the invention which has no deuterium atom as a hydrogen atom in the molecule may contain deuterium atoms in a proportion of lower than or equal to the natural abundance ratio thereof.

The fact that the proportion of deuterium atom to the sum of protium atoms and deuterium atoms is lower than or equal to the natural abundance ratio thereof can be confirmed by a nuclear magnetic resonance spectrometer.

In one embodiment, the compound represented by the formula (101) has at least one deuterium atom as a hydrogen atom in the molecule.

In one embodiment, R₂₀₁ to R₂₀₈ which are substituent R's have at least one deuterium atom.

In this specification, “having a deuterium atom as a hydrogen atom” means that the proportion of a deuterium atom to the sum of a protium atom and a deuterium atom in the relevant hydrogen atom is higher than the natural abundance ratio thereof. The fact that the proportion of a deuterium atom to the sum of a protium atom and a deuterium atom is higher than the natural abundance ratio thereof can be confirmed by a nuclear magnetic resonance spectrometer.

The compound represented by the formula (101) can be synthesized in accordance with Examples by using known alternative reactions or raw materials tailored to the target compound.

Hereinafter, specific examples of the compound represented by the formula (101) will be described, but are illustrative only, and the compound represented by the formula (101) is not limited to the following specific examples.

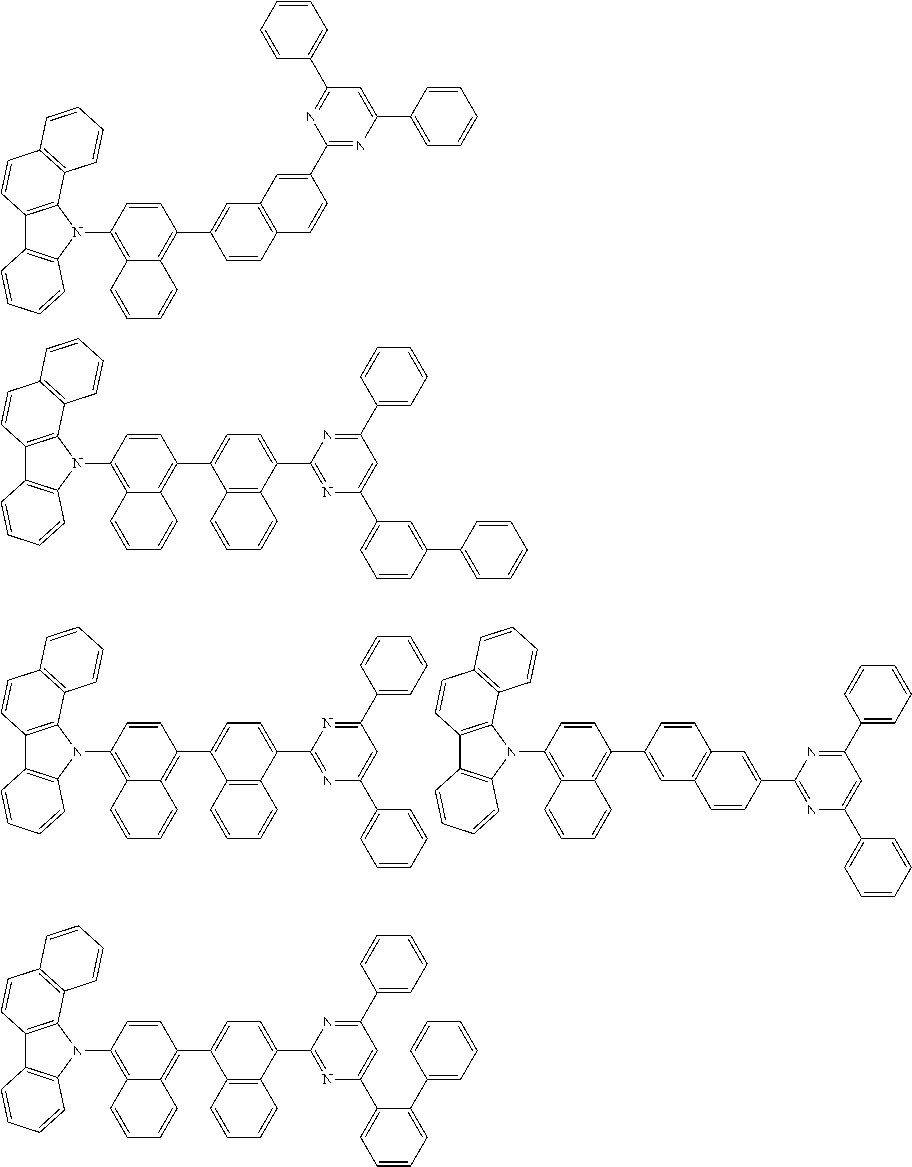

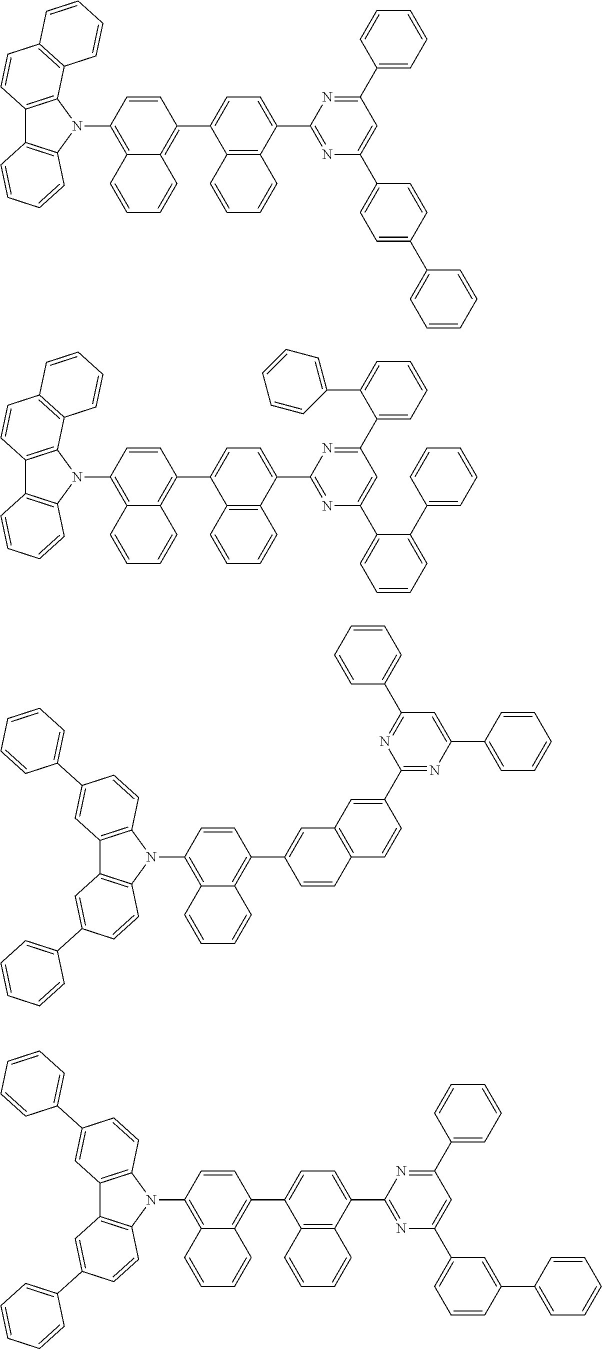

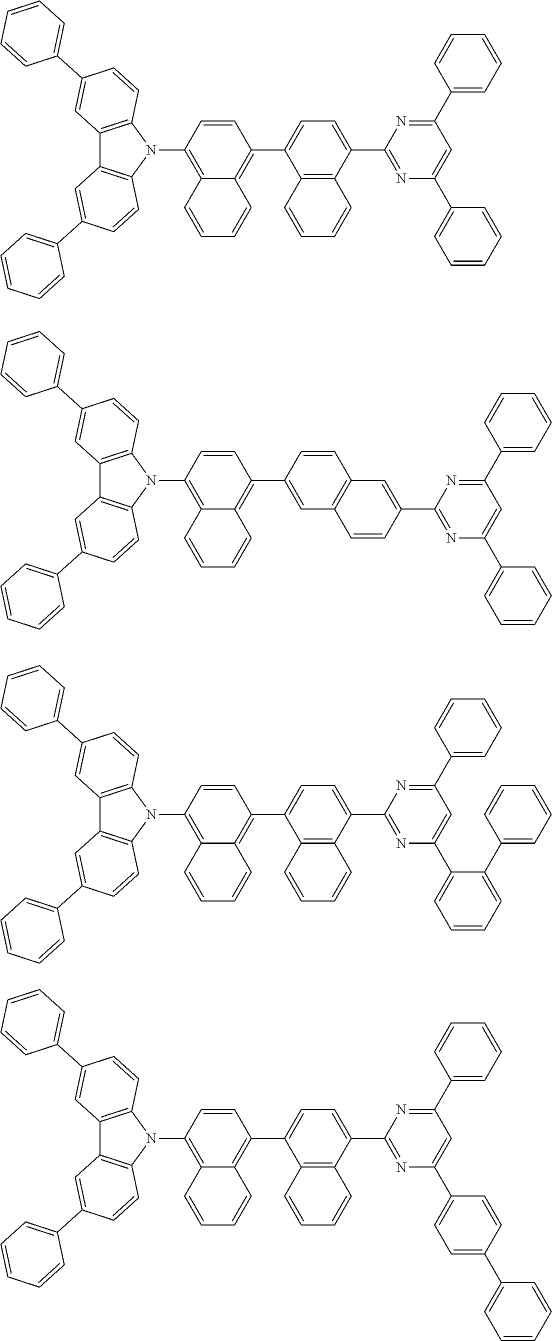

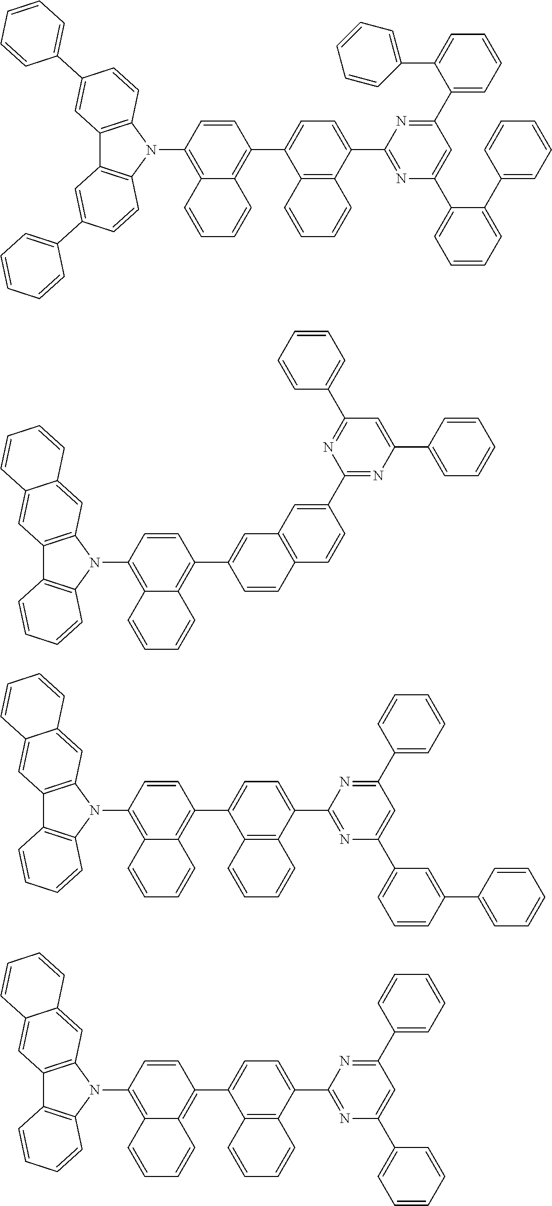

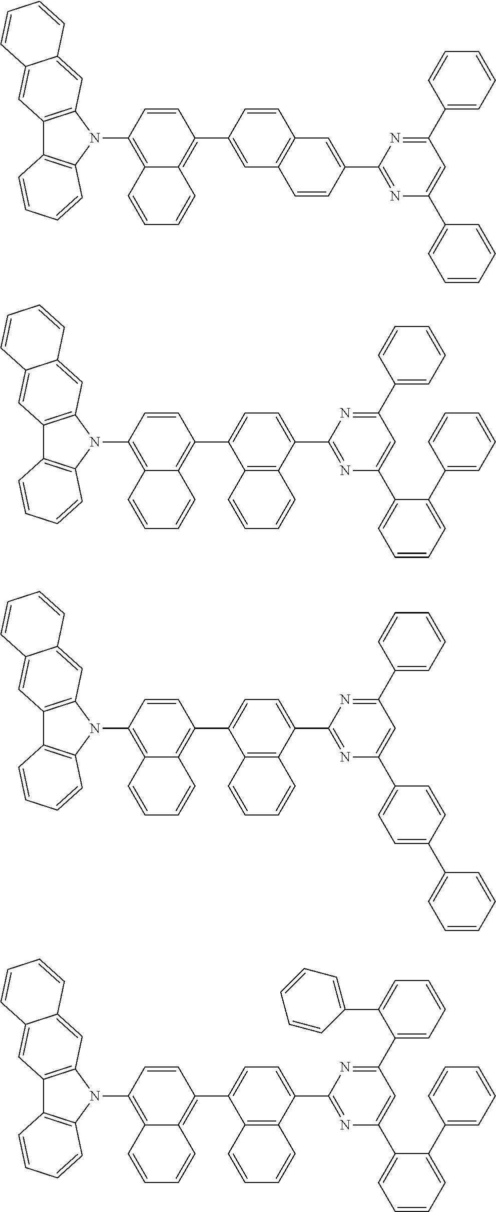 [SMILES: c1ccc(-c2cc(-c3ccccc3)nc(-c3ccc4cc(-c5ccc(-n6c7ccccc7c7cc8ccccc8cc76)c6ccccc56)ccc4c3)n2)cc1 ; c1ccc(-c2cc(-c3ccccc3-c3ccccc3)nc(-c3ccc(-c4ccc(-n5c6ccccc6c6cc7ccccc7cc65)c5ccccc45)c4ccccc34)n2)cc1 ; c1ccc(-c2ccc(-c3cc(-c4ccccc4)nc(-c4ccc(-c5ccc(-n6c7ccccc7c7cc8ccccc8cc76)c6ccccc56)c5ccccc45)n3)cc2)cc1 ; c1ccc(-c2ccccc2-c2cc(-c3ccccc3-c3ccccc3)nc(-c3ccc(-c4ccc(-n5c6ccccc6c6cc7ccccc7cc65)c5ccccc45)c4ccccc34)n2)cc1]

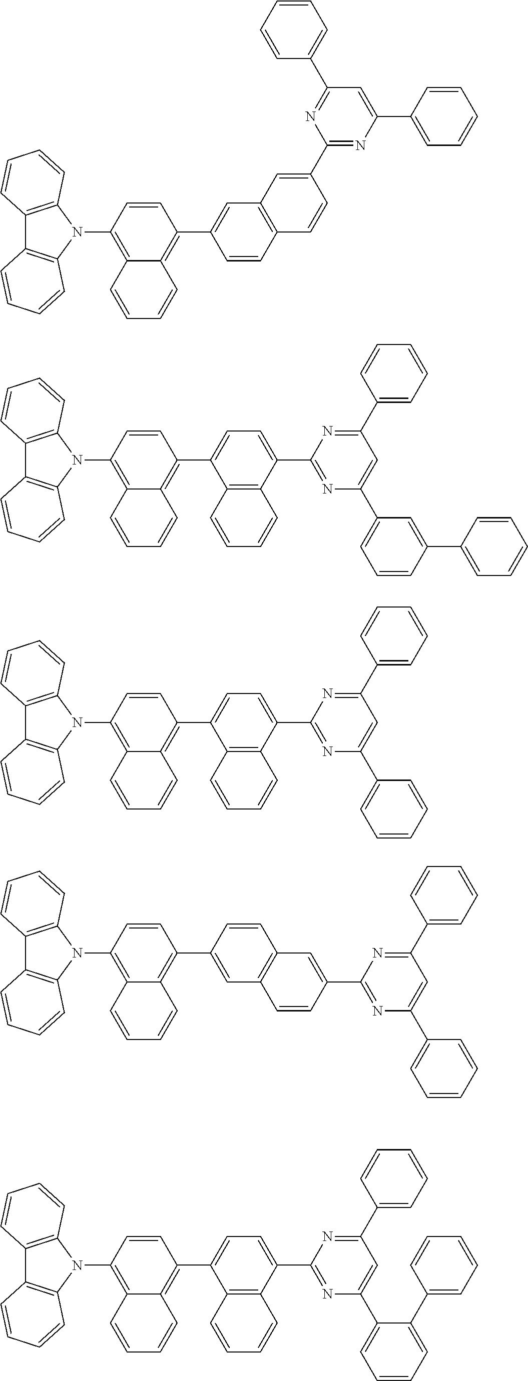

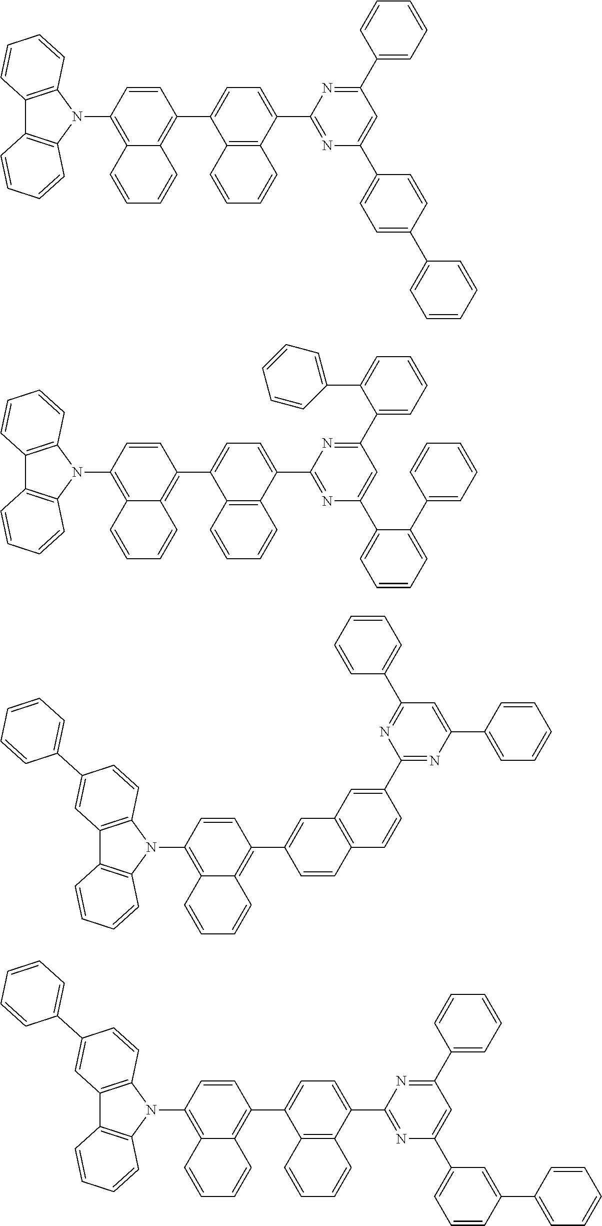

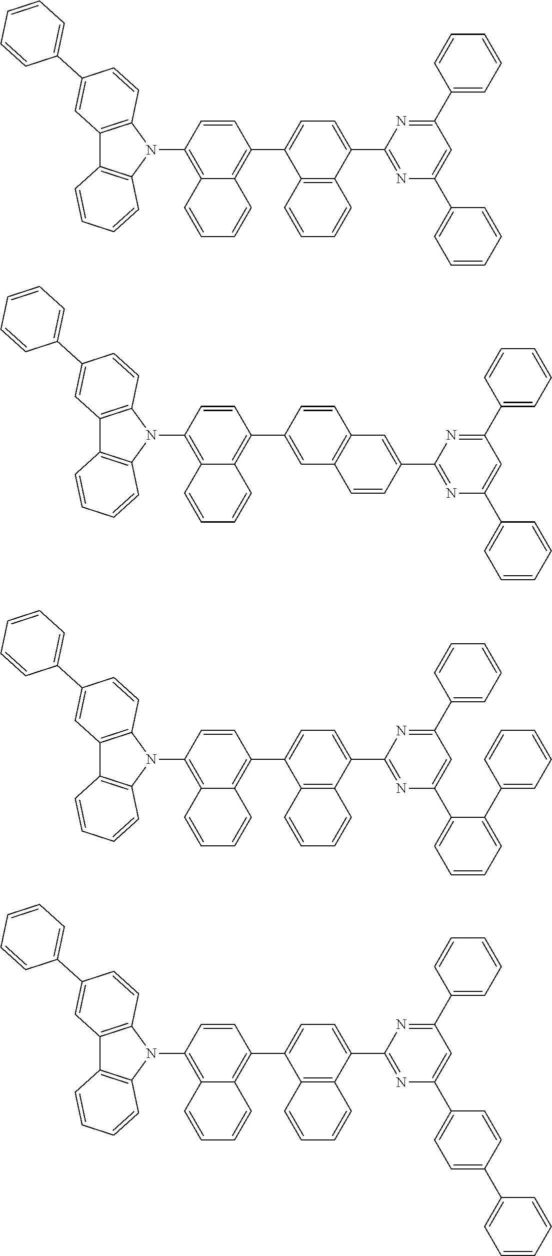 [SMILES: c1ccc(-c2ccc3c(c2)c2ccccc2n3-c2ccc(-c3ccc(-c4nc(-c5ccccc5)cc(-c5ccccc5)n4)c4ccccc34)c3ccccc23)cc1 ; c1ccc(-c2ccc3c(c2)c2ccccc2n3-c2ccc(-c3ccc4cc(-c5nc(-c6ccccc6)cc(-c6ccccc6)n5)ccc4c3)c3ccccc23)cc1 ; c1ccc(-c2ccc3c(c2)c2ccccc2n3-c2ccc(-c3ccc(-c4nc(-c5ccccc5)cc(-c5ccccc5-c5ccccc5)n4)c4ccccc34)c3ccccc23)cc1 ; c1ccc(-c2ccc(-c3cc(-c4ccccc4)nc(-c4ccc(-c5ccc(-n6c7ccccc7c7cc(-c8ccccc8)ccc76)c6ccccc56)c5ccccc45)n3)cc2)cc1]

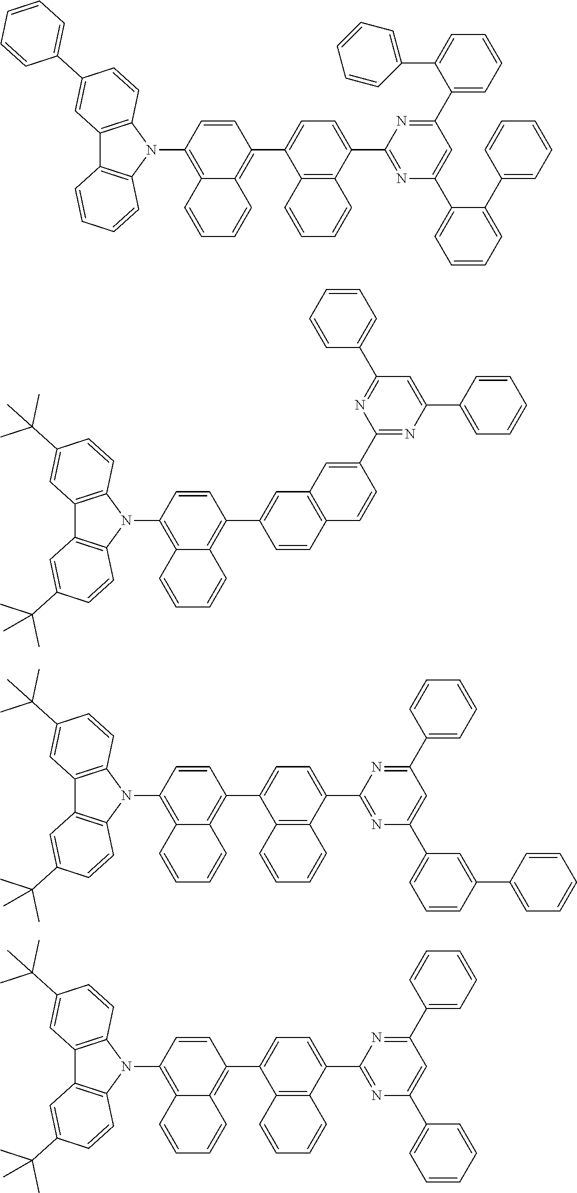

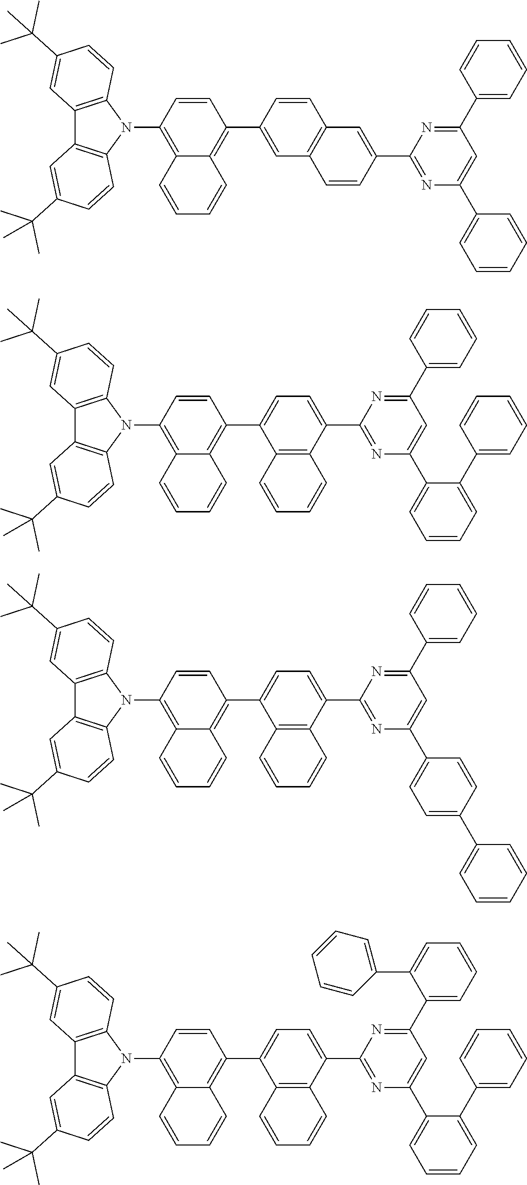

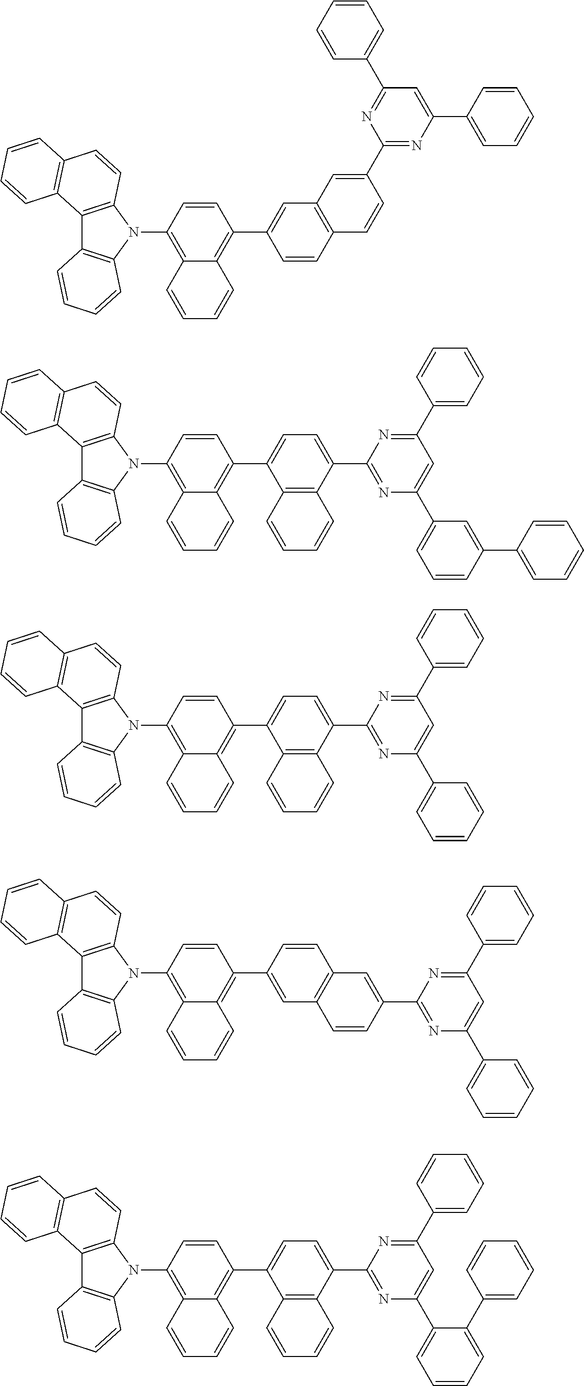

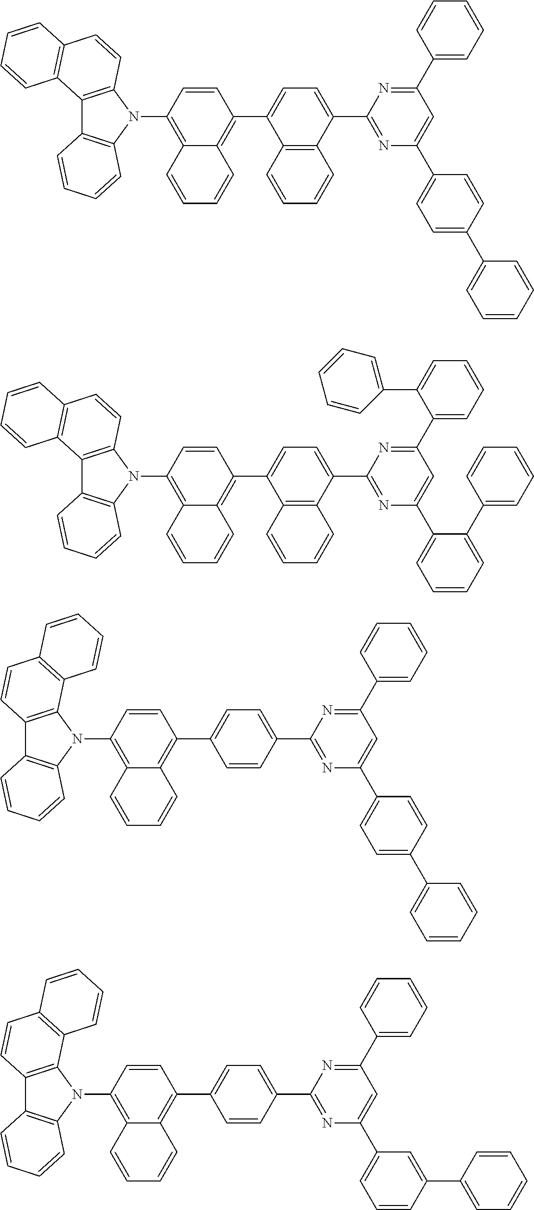

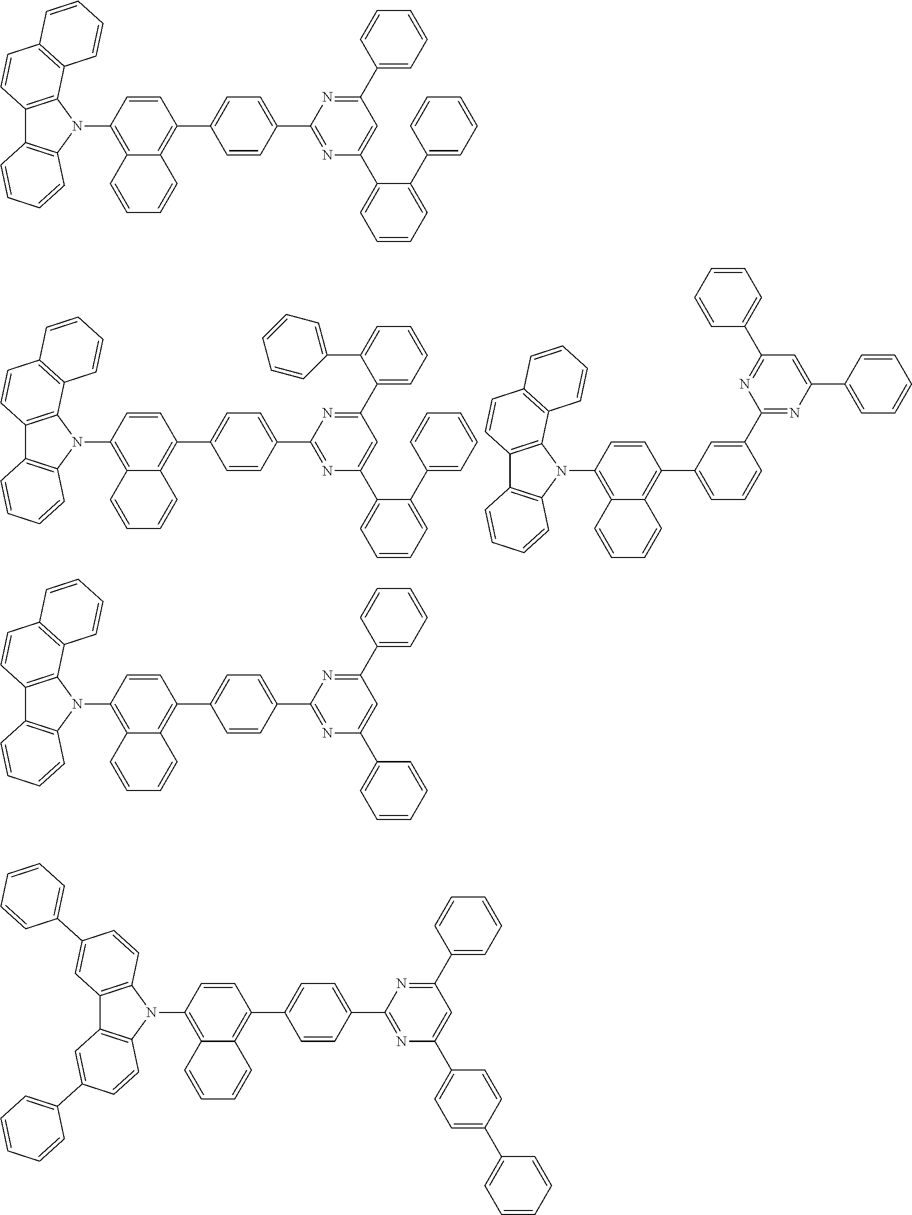

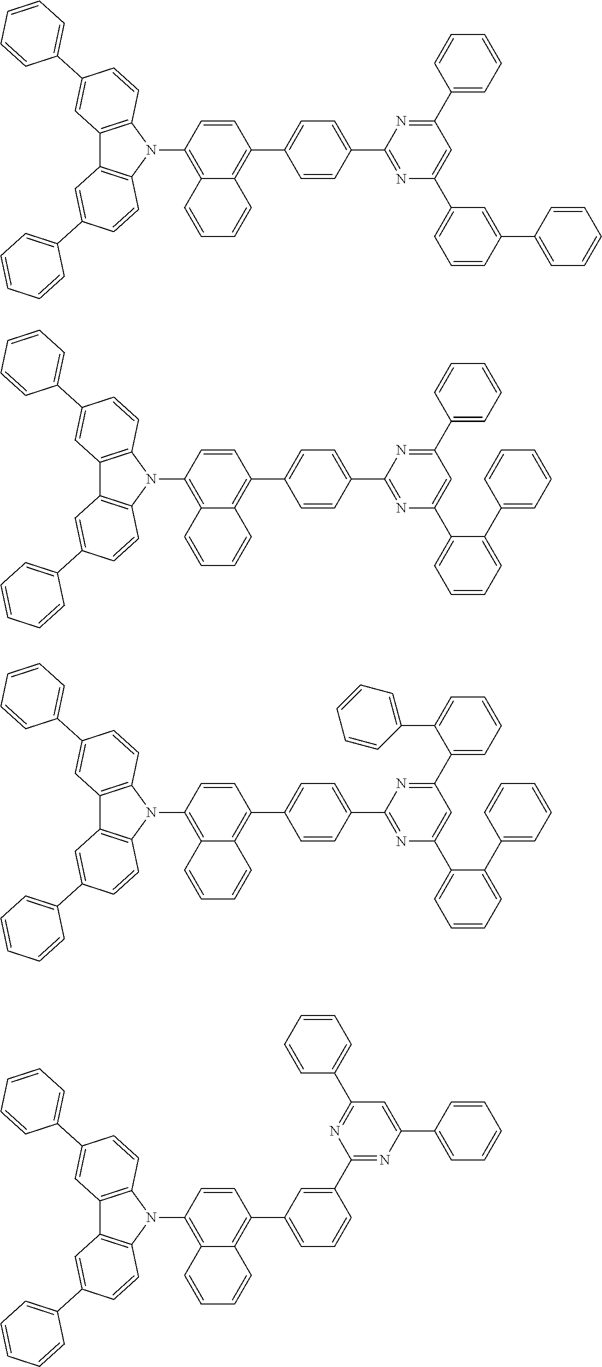

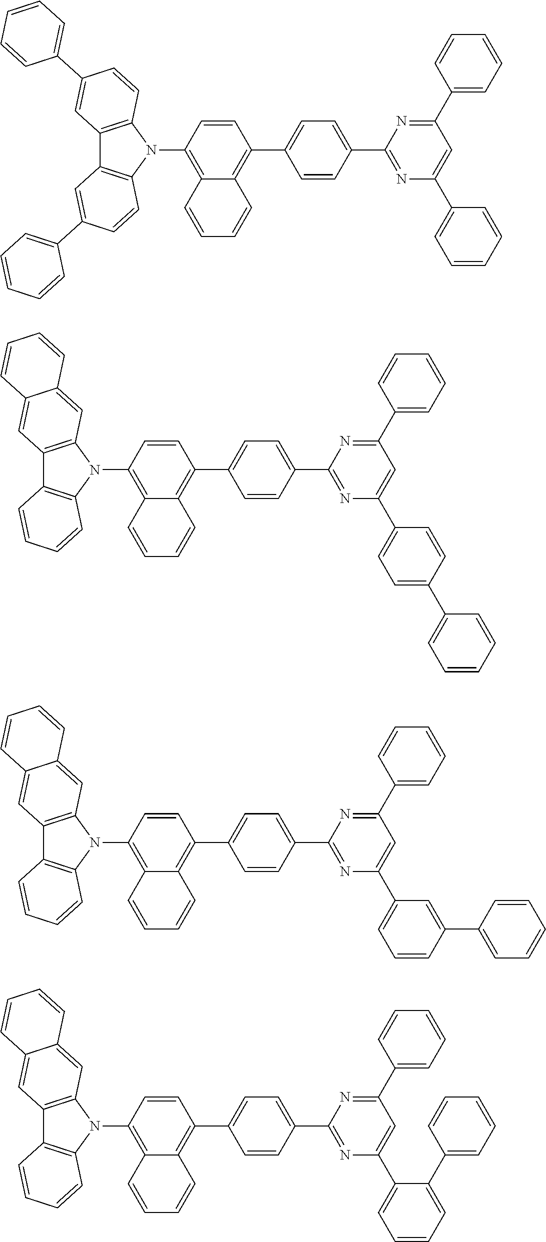

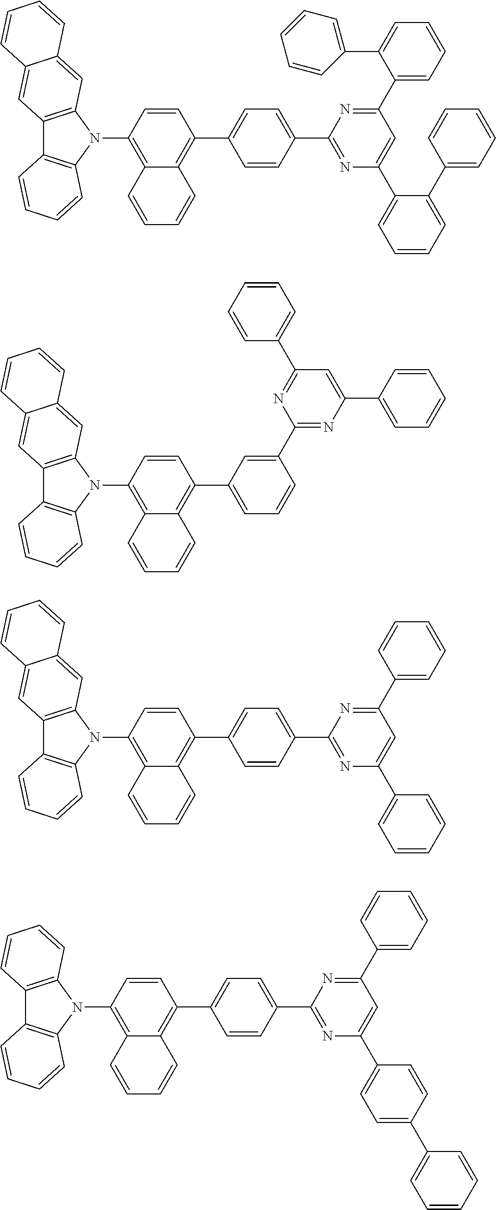

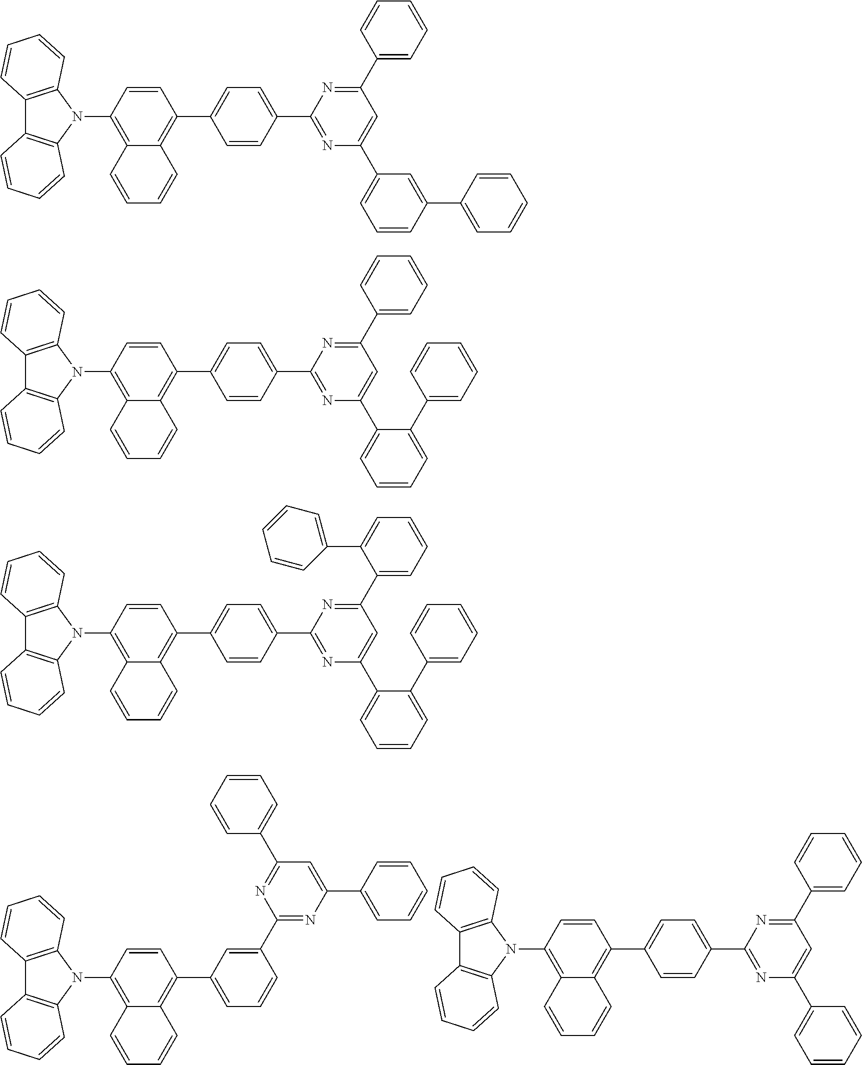

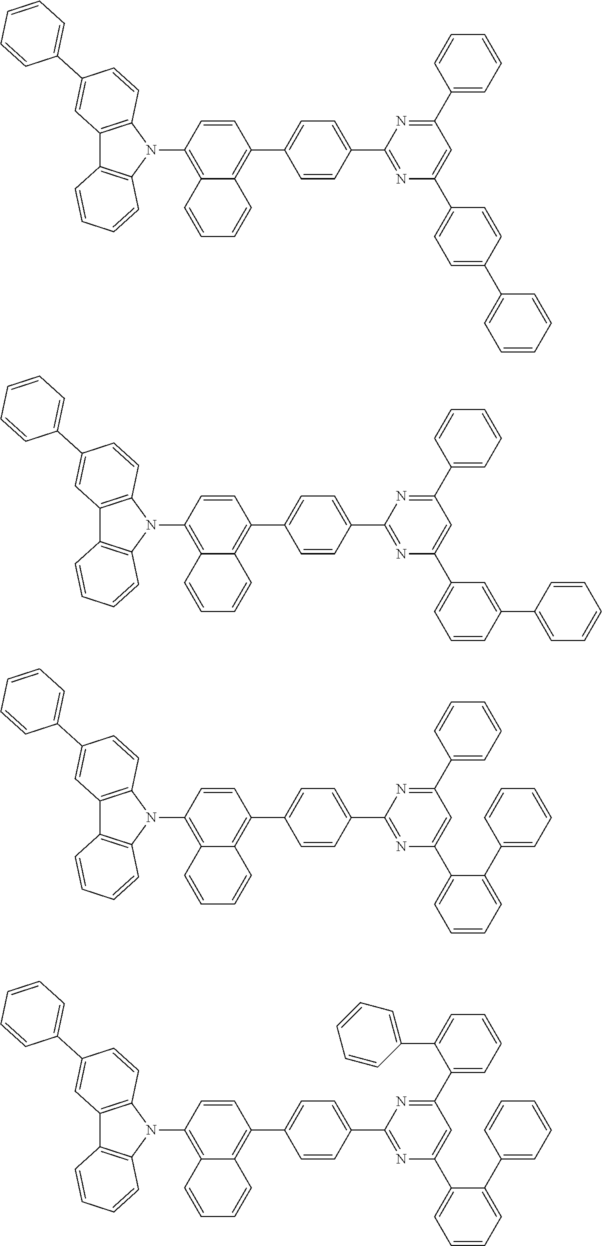 [SMILES: c1ccc(-c2ccc(-c3cc(-c4ccccc4)nc(-c4ccc(-c5ccc(-n6c7ccccc7c7cc(-c8ccccc8)ccc76)c6ccccc56)cc4)n3)cc2)cc1 ; c1ccc(-c2cccc(-c3cc(-c4ccccc4)nc(-c4ccc(-c5ccc(-n6c7ccccc7c7cc(-c8ccccc8)ccc76)c6ccccc56)cc4)n3)c2)cc1 ; c1ccc(-c2ccc3c(c2)c2ccccc2n3-c2ccc(-c3ccc(-c4nc(-c5ccccc5)cc(-c5ccccc5-c5ccccc5)n4)cc3)c3ccccc23)cc1 ; c1ccc(-c2ccc3c(c2)c2ccccc2n3-c2ccc(-c3ccc(-c4nc(-c5ccccc5-c5ccccc5)cc(-c5ccccc5-c5ccccc5)n4)cc3)c3ccccc23)cc1]

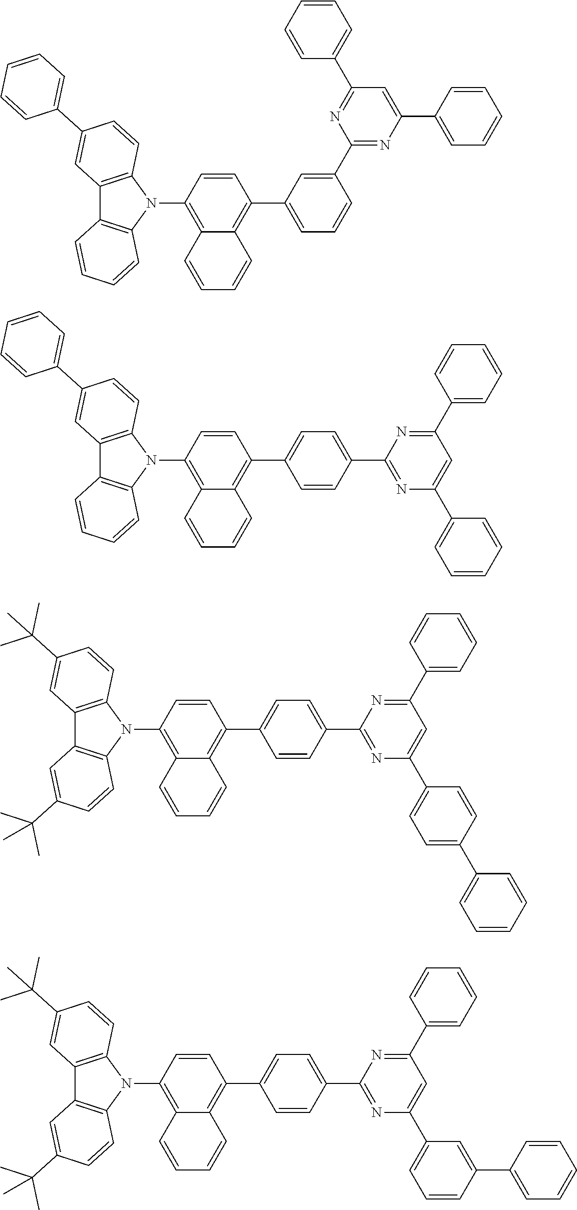

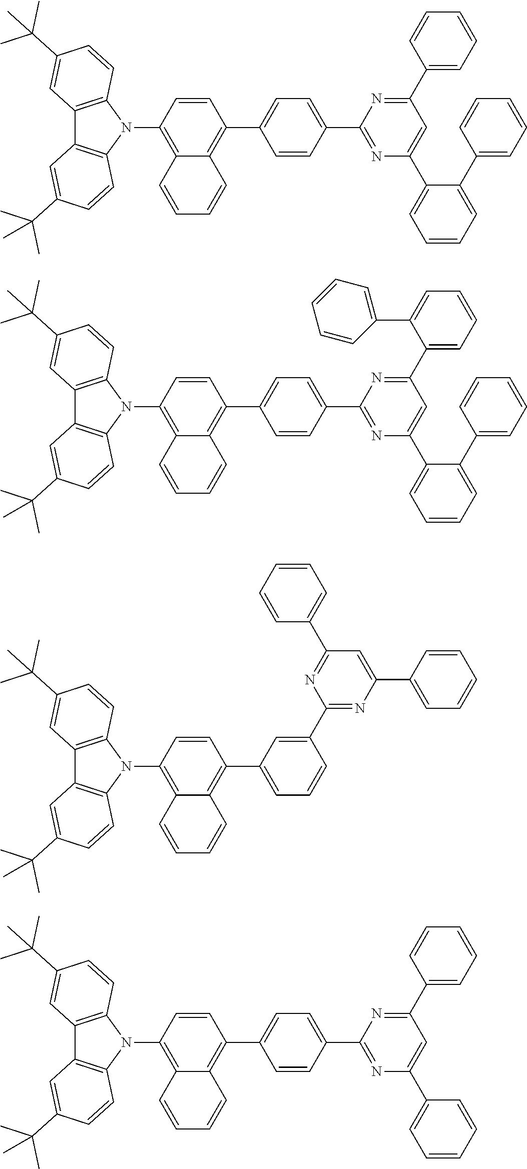

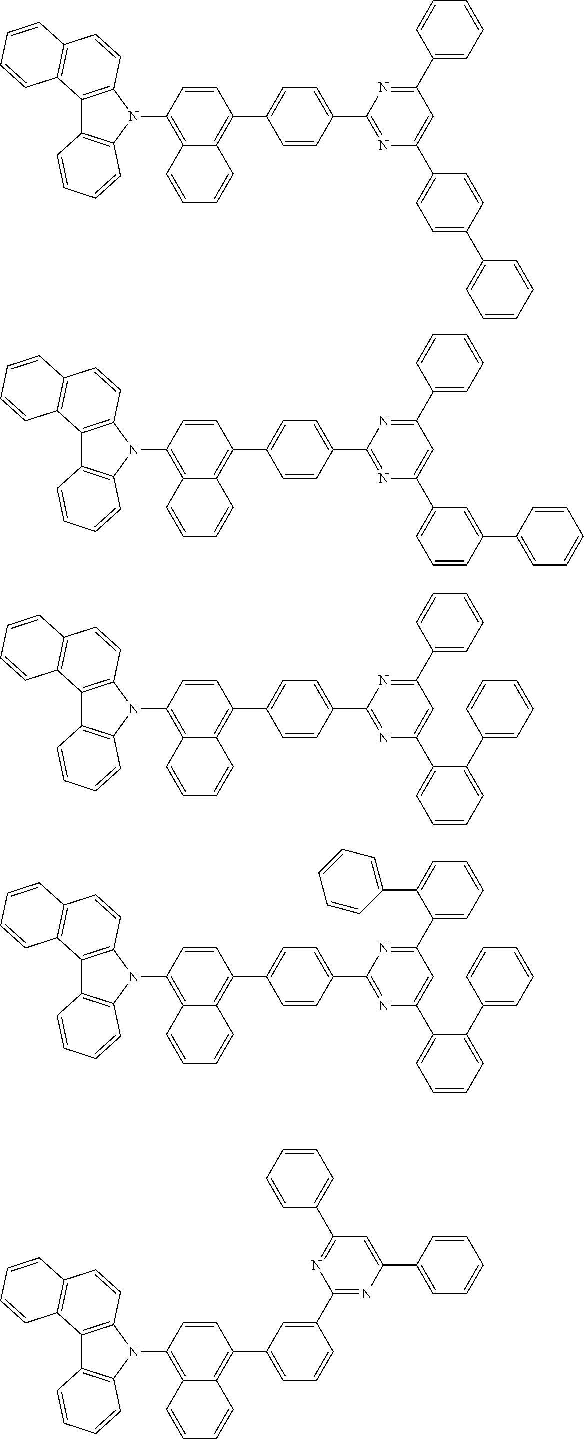

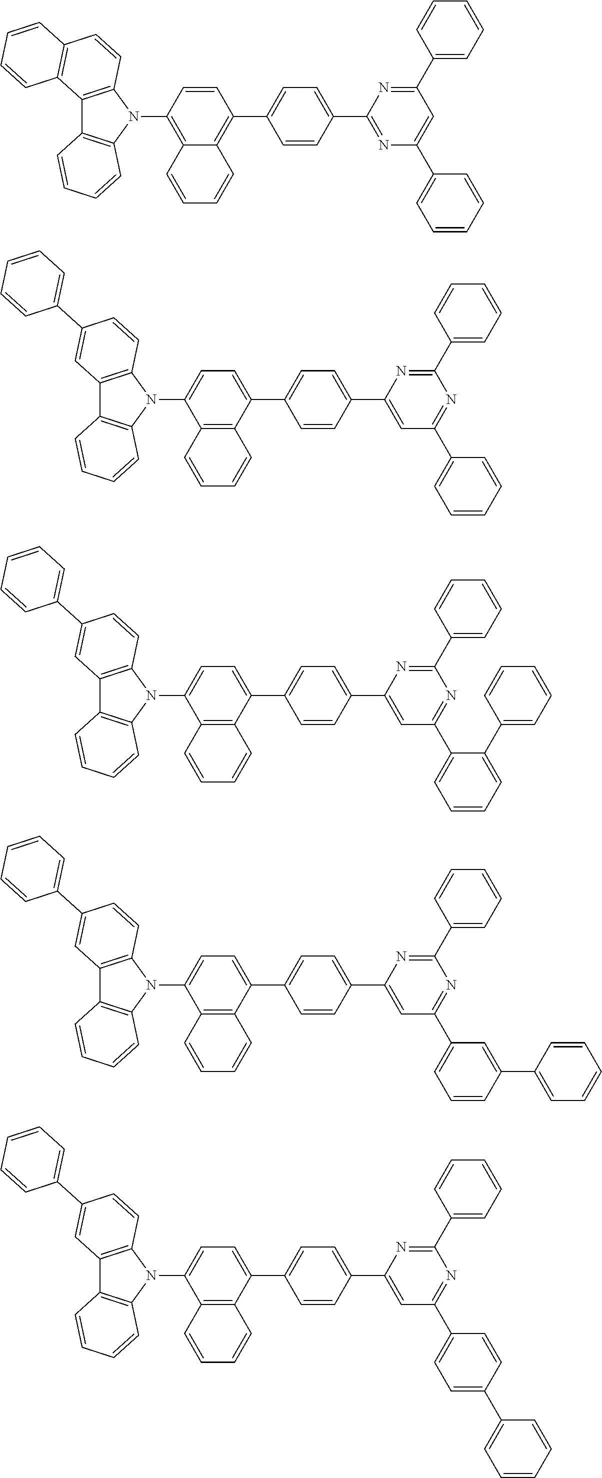 [SMILES: c1ccc(-c2cc(-c3ccccc3)nc(-c3ccc(-c4ccc(-n5c6ccccc6c6c7ccccc7ccc65)c5ccccc45)cc3)n2)cc1 ; c1ccc(-c2ccc3c(c2)c2ccccc2n3-c2ccc(-c3ccc(-c4cc(-c5ccccc5)nc(-c5ccccc5)n4)cc3)c3ccccc23)cc1 ; c1ccc(-c2ccc3c(c2)c2ccccc2n3-c2ccc(-c3ccc(-c4cc(-c5ccccc5-c5ccccc5)nc(-c5ccccc5)n4)cc3)c3ccccc23)cc1 ; c1ccc(-c2cccc(-c3cc(-c4ccc(-c5ccc(-n6c7ccccc7c7cc(-c8ccccc8)ccc76)c6ccccc56)cc4)nc(-c4ccccc4)n3)c2)cc1 ; c1ccc(-c2ccc(-c3cc(-c4ccc(-c5ccc(-n6c7ccccc7c7cc(-c8ccccc8)ccc76)c6ccccc56)cc4)nc(-c4ccccc4)n3)cc2)cc1]

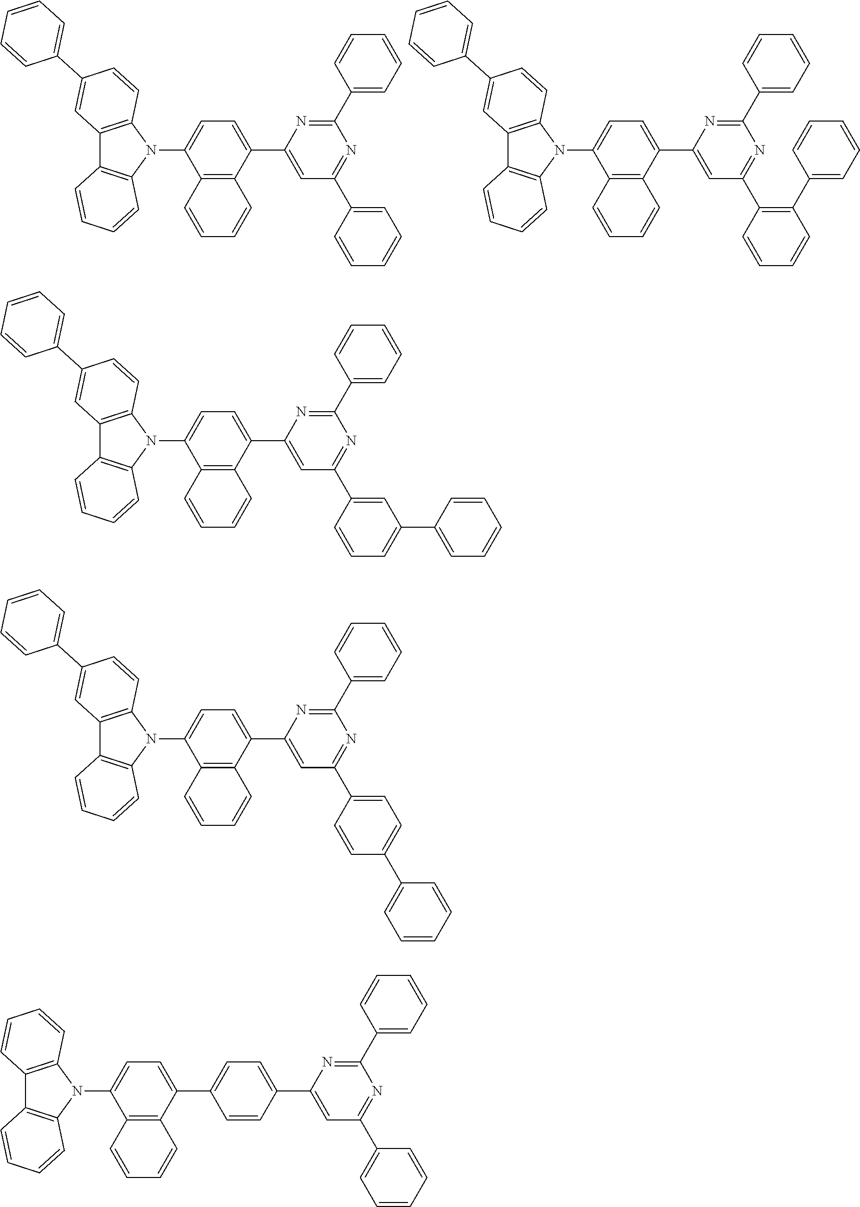

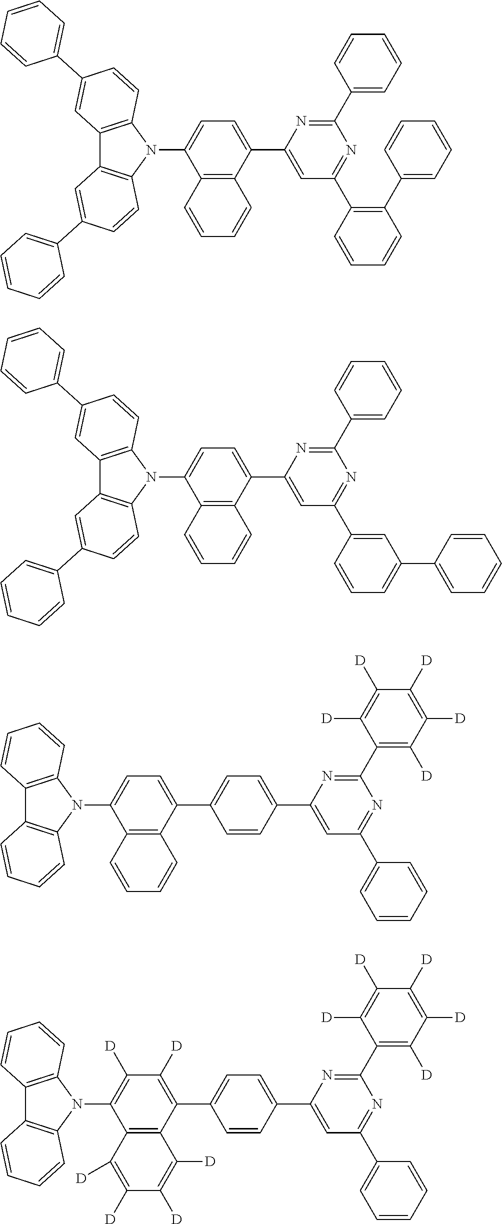

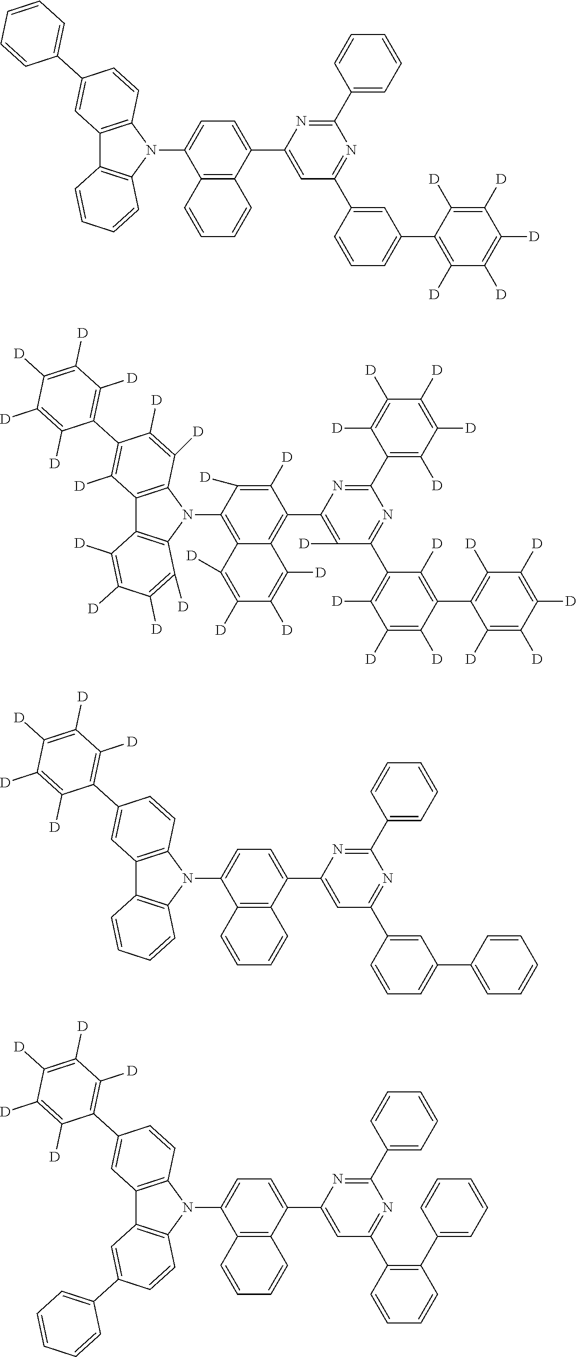

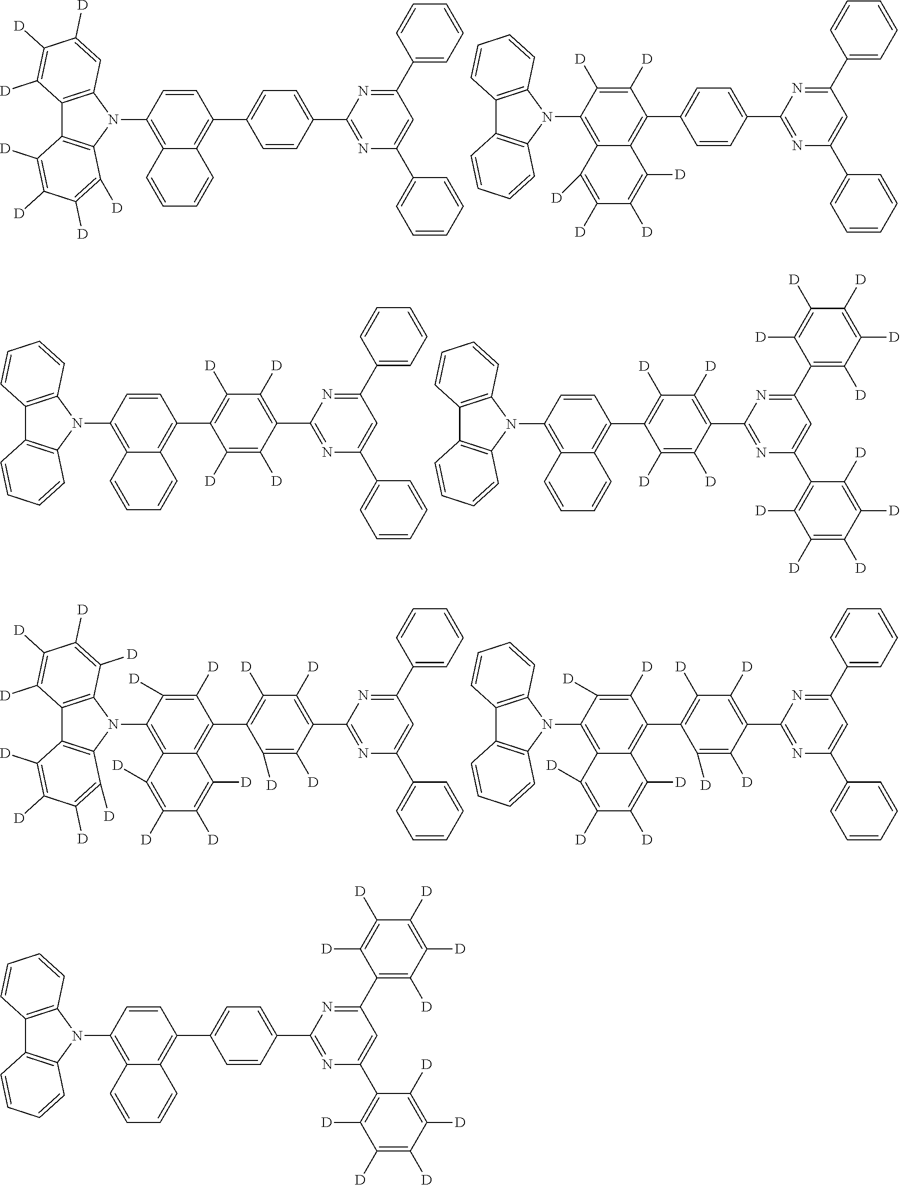

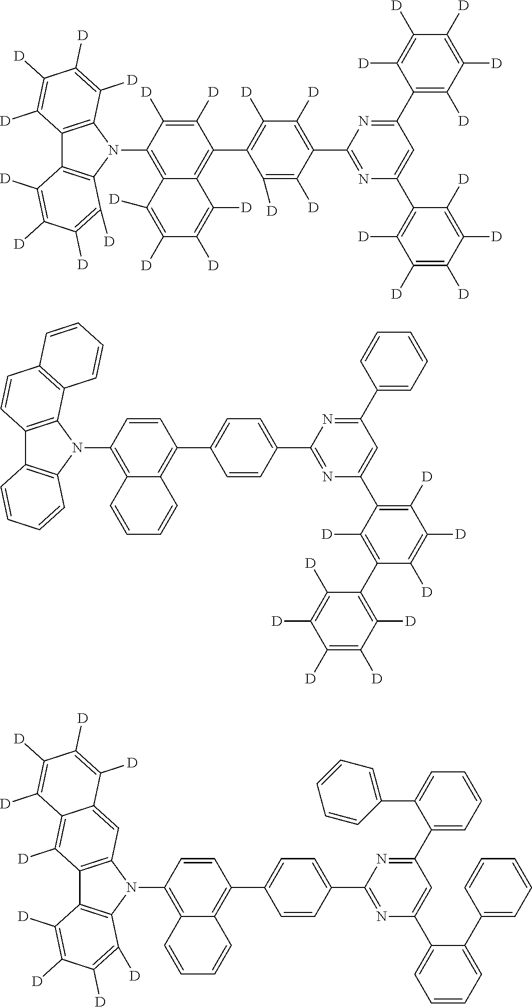

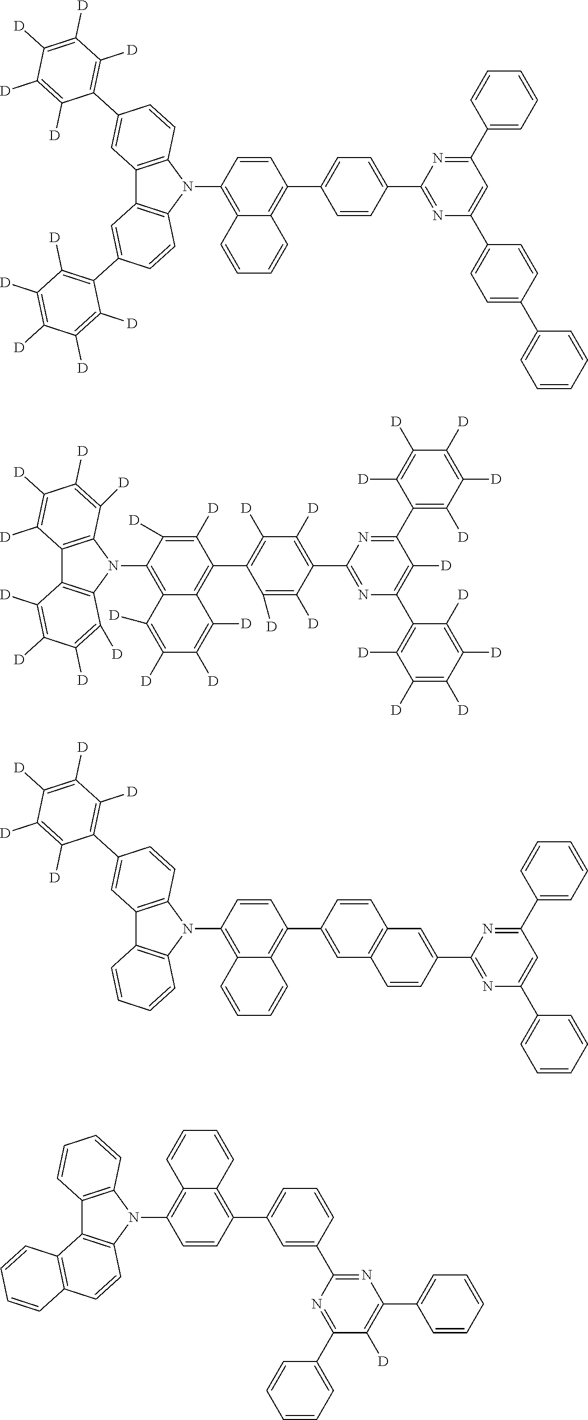

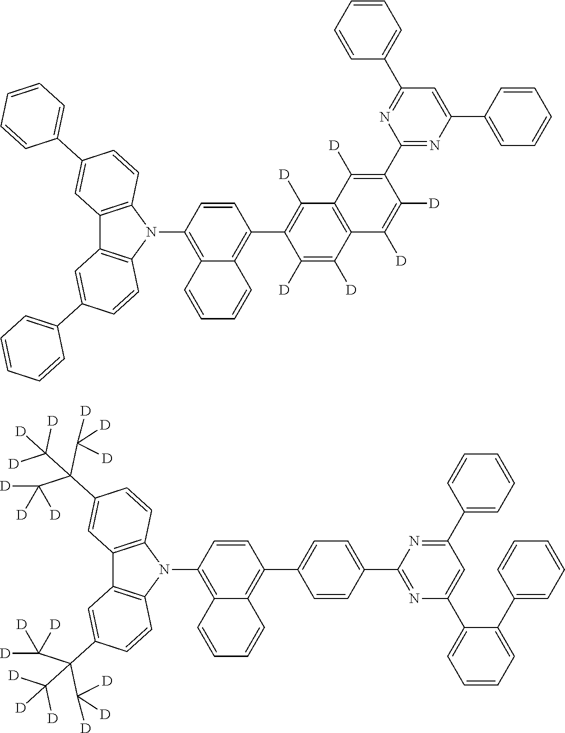

### [Material for Organic Electroluminescence Device]

The compound according to one aspect of the invention is useful as a material for an organic EL device, and is useful, for example, as a material used for an electron-transporting region of an organic EL device.

### [Organic EL Device]

The organic EL device according to one aspect of the invention will be described.

An organic EL device according to one aspect of the invention has a cathode, an anode, and one or two or more organic layers arranged between the cathode and anode, wherein at least one of the organic layers includes the compound (represented by the formula (1)) according to one aspect of the invention.

In one embodiment, an organic EL device according to one aspect of the invention has an anode, an emitting layer, an electron-transporting region, and a cathode in this order, and the electron-transporting region includes the compound (represented by the formula (1)) according to one aspect of the invention.

In one embodiment, the electron-transporting region has a first layer (also referred to as a “first electron-transporting layer” or “hole barrier layer”) and a second layer (also referred to as a “second electron-transporting layer”) in this order from the emitting layer side, and the first layer contains the compound represented by the formula (1). As the above-described second layer, in this case, an electron-transporting layer configuration described later can be applied.

As the typical device configuration of the organic EL device, configurations in which the following structures are stacked on a substrate are exemplified:
(1) an anode/an emitting layer/an electron-transporting region/a cathode
(2) an anode/a hole-transporting region/an emitting layer/an electron-transporting region/a cathode
   “/” indicates that the layers are stacked adjacent to each other.

The electron-transporting region is generally composed of one or more layers selected from an electron-injecting layer and an electron-transporting layer. The hole-transporting region is generally composed of one or more layers selected from a hole-injecting layer and a hole-transporting layer.

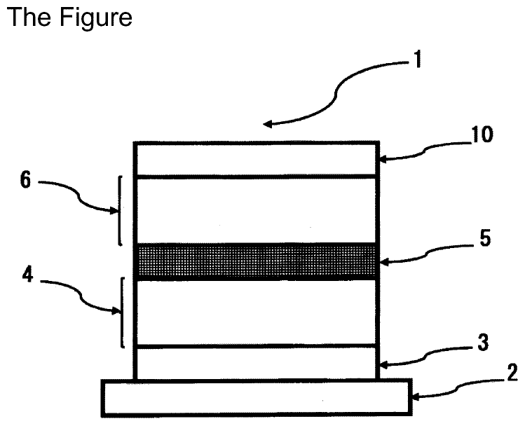

The schematic configuration of the organic EL device of one aspect of the invention will be described referring to FIG. 1.

An organic EL device 1 according to one aspect of the invention comprises a substrate 2, an anode 3, an emitting layer 5, a cathode 10, a hole-transporting region 4 between the anode 3 and the emitting layer 5, and an electron-transporting region 6 between the emitting layer 5 and the cathode 10.

Members which can be used in the organic EL device according to one aspect of the invention, materials for forming each layer, other than the above-mentioned compounds, and the like, are described.

### (Substrate)

A substrate is used as a support of an emitting device. As the substrate, glass, quartz, plastic or the like can be used, for example. Further, a flexible substrate may be used. The “flexible substrate” means a bendable (flexible) substrate, and specific examples thereof include a plastic substrate formed of polycarbonate, polyvinyl chloride, or the like.

### (Anode)

For the anode formed on the substrate, metals, alloys, electrically conductive compounds, mixtures thereof, and the like, which have a large work function (specifically 4.0 eV or more) are preferably used. Specific examples thereof include indium oxide-tin oxide (ITO: Indium Tin Oxide), indium oxide-tin oxide containing silicon or silicon oxide, indium oxide-zinc oxide, tungsten oxide, indium oxide containing zinc oxide, graphene, and the like. In addition thereto, specific examples thereof include gold (Au), platinum (Pt), a nitride of a metallic material (for example, titanium nitride), and the like.

### (Hole-Injecting Layer)

The hole-injecting layer is a layer containing a substance having a high hole-injecting property. As the substance having high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (oligomers, dendrimers, polymers, or the like) and the like can be used.

### (Hole-Transporting Layer)

The hole-transporting layer is a layer containing a substance having a high hole-transporting property. For the hole-transporting layer, an aromatic amine compound, a carbazole derivative, an anthracene derivative, or the like can be used. A polymer compound such as poly(N-vinylcarbazole)(abbreviation: PVK) and poly(4-vinyltriphenylamine)(abbreviation: PVTPA) can also be used. Provided that a substance other than the above-described substances may be used as long as the substance has a higher hole-transporting property than an electron-transporting property. The layer containing the substance having high hole-transporting property may be not only a single layer, but also layers in which two or more layers formed of the above-described substances are stacked.

### (Guest (Dopant) Material of Emitting Layer)

The emitting layer is a layer containing a substance having a high emitting property, and various materials can be used for the emitting layer. For example, as the substance having a high emitting property, a fluorescent compound which emits fluorescence, and a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for the emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for the emitting layer, aromatic amine derivatives and the like can be used. As a red fluorescent emitting material which can be used for the emitting layer, tetracene derivatives, diamine derivatives and the like can be used.

As a blue phosphorescent emitting material which can be used for the emitting layer, metal complexes such as iridium complexes, osmium complexes, platinum complexes and the like are used. As a green phosphorescent emitting material which can be used for the emitting layer, iridium complexes and the like are used. As a red phosphorescent emitting material which can be used for the emitting layer, metal complexes such as iridium complexes, platinum complexes, terbium complexes, europium complexes and the like can be used.

### (Host Material for Emitting Layer)

The emitting layer may have a constitution in which the substance having a high emitting property (guest material) is dispersed in another substance (host material). As such another substance for dispersing the substance having a high emitting property, a variety of substances can be used, and it is preferable to use such another substance having a higher lowest unoccupied orbital level (LUMO level) and a lower highest occupied orbital level (HOMO level) than the substance having a high emitting property.

As such another substance for dispersing the substance having a high emitting property (host material), 1) metal complexes such as aluminum complexes, beryllium complexes, zinc complexes, and the like; 2) heterocyclic compounds such as oxadiazole derivatives, benzimidazole derivatives, phenanthroline derivatives, and the like; 3) fused aromatic compounds such as carbazole derivatives, anthracene derivatives, phenanthrene derivatives, pyrene derivatives, chrysene derivatives, and the like; and 4) aromatic amine compound such as triarylamine derivatives, fused polycyclic aromatic amine derivatives, and the like are used.

### (Electron-Transporting Layer)

The electron-transporting layer is a layer containing a substance having a high electron-transporting property. For the electron-transporting layer, 1) metal complexes such as aluminum complexes, beryllium complexes, zinc complexes, and the like; 2) heteroaromatic compounds such as imidazole derivatives, benzimidazole derivatives, azine derivatives, carbazole derivatives, phenanthroline derivatives, and the like; and 3) polymer compounds can be used.

In one embodiment of the invention, the electron-transporting layer may or may not contain other materials described above in addition to the compound (represented by the formula (1)) according to one aspect of the invention.

### (Electron-Injecting Layer)

The electron-injecting layer is a layer containing a substance having a high electron-injecting property. For the electron-injecting layer, lithium (Li), ytterbium (Yb), lithium fluoride (LiF), cesium fluoride (CsF), calcium fluoride (CaF₂), metal complexes such as 8-hydroxyquinolinolato-lithium (Liq), alkaline metals, alkaline earth metals, or compounds thereof such as lithium oxide (LiOₓ) can be used.

### (Cathode)

For the cathode, metals, alloys, electrically conductive compounds, mixtures thereof, and the like, which have a small work function (specifically, 3.8 eV or lower) are preferably used. Specific examples of such a cathode material include elements each belonging to Group 1 or Group 2 of the Periodic Table of the Elements, i.e., alkali metals such as lithium (Li) and cesium (Cs), alkaline earth metals such as magnesium (Mg), calcium (Ca) and strontium (Sr), and alloys containing these metals (e.g., MgAg and AILi); rare earth metals such as europium (Eu) and ytterbium (Yb), and alloys containing these metals.

The cathode is generally formed by a vacuum deposition method or a sputtering method. Further, in the case of using a silver paste or the like, a coating method, an inkjet method, or the like can be employed.

In the case where the electron-injecting layer is provided, a cathode can be formed using various electrically conductive materials such as aluminum, silver, ITO, graphene, indium oxide-tin oxide containing silicon or silicon oxide, regardless of the work function value.

In the organic EL device according to one aspect of the invention, the thickness of each layer is not particularly limited, but is generally preferable that the thickness be generally in the range of several nm to 1 μm in order to decrease defects such as pinholes, to suppress applied voltages to be low, and to increase luminous efficiency.

In the organic EL device according to one aspect of the invention, the method for forming each of the layers is not particularly limited. Conventionally-known forming methods such as a vacuum deposition method, a spin coating method and the like, can be used. Each of the layers such as the emitting layer can be formed by a known method such as a vacuum deposition method, a molecular beam deposition method (MBE method), or an application method such as a dipping method, a spin coating method, a casting method, a bar coating method, or a roll coating method, using a solution prepared by dissolving the material in a solvent.

### [Electronic Apparatus]

The electronic apparatus according to one aspect of the invention is equipped with the organic EL device according to one aspect of the invention.

Specific examples of the electronic apparatus include display components such as an organic EL panel module, and the like; display devices such as a television, a cellular phone, a personal computer, and the like; and emitting devices such as a light, a vehicular lamp, and the like.

### EXAMPLES

### <Compound>

Compounds represented by the formula (101) used in the fabrication of organic EL devices of Examples are shown below.

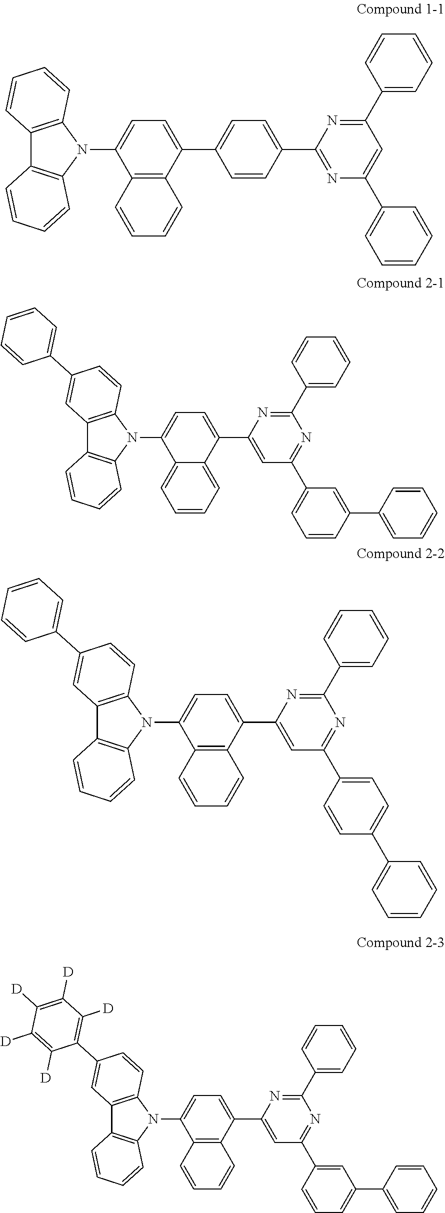 [SMILES: c1ccc(-c2cc(-c3ccccc3)nc(-c3ccc(-c4ccc(-n5c6ccccc6c6ccccc65)c5ccccc45)cc3)n2)cc1 ; c1ccc(-c2cccc(-c3cc(-c4ccc(-n5c6ccccc6c6cc(-c7ccccc7)ccc65)c5ccccc45)nc(-c4ccccc4)n3)c2)cc1 ; c1ccc(-c2ccc(-c3cc(-c4ccc(-n5c6ccccc6c6cc(-c7ccccc7)ccc65)c5ccccc45)nc(-c4ccccc4)n3)cc2)cc1 ; [2H]c1c([2H])c([2H])c(-c2ccc3c(c2)c2ccccc2n3-c2ccc(-c3cc(-c4cccc(-c5ccccc5)c4)nc(-c4ccccc4)n3)c3ccccc23)c([2H])c1[2H]]

A comparative compound used in the fabrication of organic EL devices of Comparative Example is shown below.

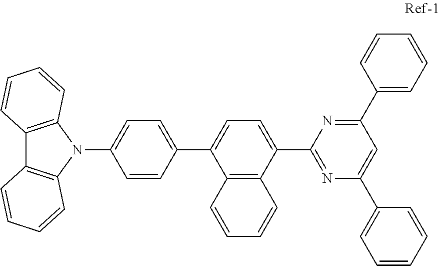 [SMILES: c1ccc(-c2cc(-c3ccccc3)nc(-c3ccc(-c4ccc(-n5c6ccccc6c6ccccc65)cc4)c4ccccc34)n2)cc1]

Other compounds used in the fabrication of organic EL devices of Examples and Comparative Example are shown below.

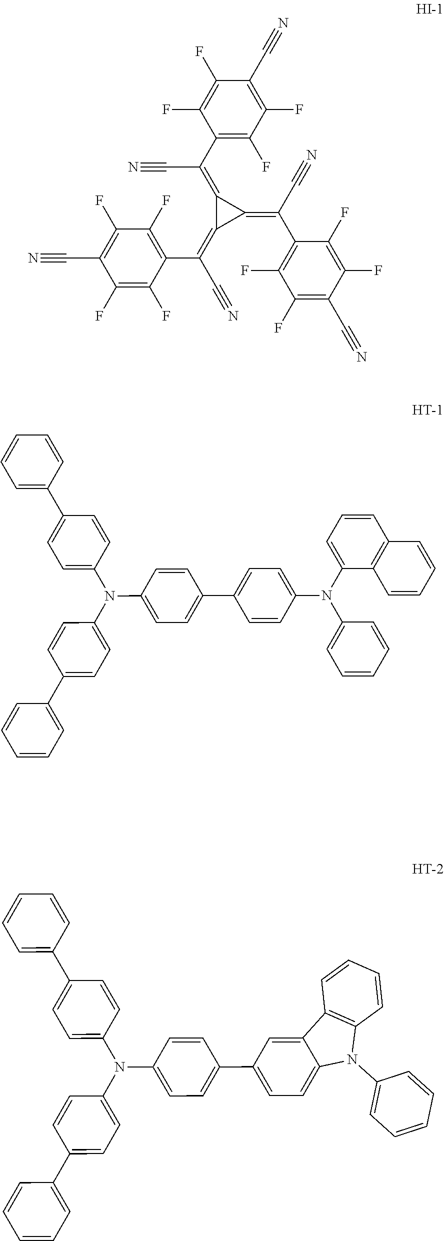 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccccc5)c5cccc6ccccc56)cc4)cc3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

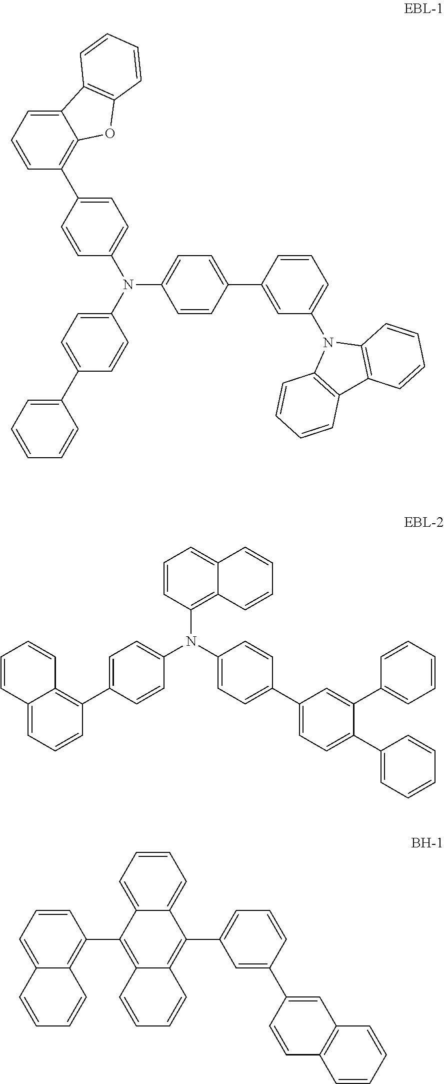

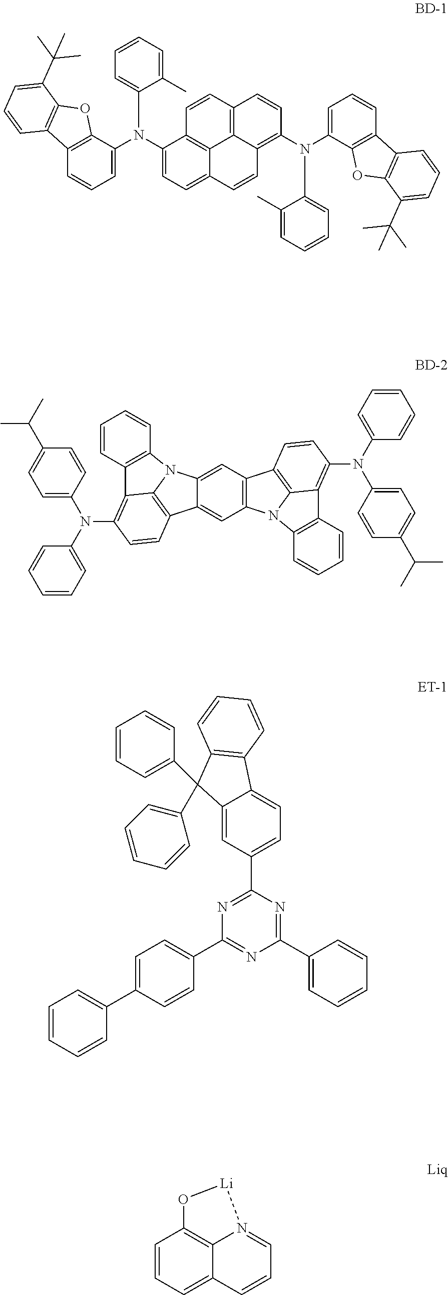

### <Fabrication 1 of Organic EL Device>

An organic EL device was fabricated as follows.

### Example 1

A 25 mm×75 mm×1.1 mm-thick glass substrate with an ITO transparent electrode (anode)(manufactured by GEOMATEC) was subjected to ultrasonic cleaning in isopropyl alcohol for 5 minutes, and then subjected to UV-ozone cleaning for 30 minutes. The thickness of the ITO film was 130 nm.

The glass substrate with a transparent electrode after cleaning was mounted on a substrate holder of a vacuum vapor deposition apparatus. First, Compounds HT-1 and HI-1 were co-deposited on the surface of the substrate on which the transparent electrode has been formed to cover the transparent electrode, such that the proportion of Compound HI-1 was 3% by mass. Thus, a first hole-transporting layer having a thickness of 10 nm was formed.

On the first hole-transporting layer, Compound HT-1 was deposited to form a second hole-transporting layer having a film thickness of 80 nm.

On the second hole-transporting layer, Compound EBL-1 was deposited to form a third hole-transporting layer (also referred to as “electron barrier layer”) having a film thickness of 5 nm.

On the third hole-transporting layer, Compound BH-1 (host material) and Compound BD-1 (dopant material) were co-deposited such that the proportion of Compound BD-1 was 4% by mass, to form an emitting layer having a film thickness of 25 nm.

On the emitting layer, Compound 1-1 was deposited to form a first electron-transporting layer (also referred to as “hole barrier layer”) having a film thickness of 5 nm.

On the first electron-transporting layer, Compound ET-1 and Liq were co-deposited such that the proportion of Liq was 50% by mass, to form a second electron-transporting layer having a film thickness of 20 nm.

Metal Yb was deposited on the second electron-transporting layer to form an electron-injecting layer having a film thickness of 1 nm.

Metal Al was deposited on the electron-injecting layer to form a cathode having a film thickness of 50 nm.

The device configuration of the organic EL device of Example 1 is schematically described as follows:
ITO(130)/HT-1:HI-1(10:3%)/HT-1(80)/EBL-1(5)/BH-1:BD-1(25:4%)/Compound1-1(5)/ET-1:Liq(20:50%)/Yb(1)/Al(50)

Numerical values in parentheses indicate film thickness (unit: nm). In addition, the numerical values expressed as percentages in parentheses indicate the proportion (% by mass) of the latter compound in the corresponding layer.

### Comparative Example 1

An organic EL device was fabricated in the same manner as in Example 1, except that Compound Ref-1 was used in place of Compound 1-1 for the formation of the first electron-transporting layer.

### <Evaluation 1 of Organic EL Device>

The organic EL devices fabricated in Example 1 and Comparative Example 1 were evaluated for external quantum efficiency as follows. The results are shown in Table 1.

### External Quantum Efficiency

A voltage was applied to the organic EL device such that the current density became 10 mA/cm², and EL emission spectrum was measured by a spectral radiance meter CS-2000 (manufactured by KONICA MINOLTA, INC.). The external quantum efficiency EQE (%) was calculated from the obtained spectral radiance spectrum. In Tables 1, EQE represents as a value relative to the value of Comparative Example 1 being taken as 100.

TABLE 1

| | | EQE relative value |
|---|---|---|
| | First | (Relative to EQE value of |
| | electron-transporting | Comparative Example 1 being |
| | layer | taken as 100) |
| Example 1 | Compound 1-1 | 106 |
| Comparative | Compound Ref-1 | 100 |
| Example 1 | | |

### <Fabrication 2 of Organic EL Device>

### Example 2

An organic EL device was fabricated as follows.

A 25 mm×75 mm×1.1 mm-thick glass substrate with an ITO transparent electrode (anode)(manufactured by GEOMATEC) was subjected to ultrasonic cleaning in isopropyl alcohol for 5 minutes, and then subjected to UV-ozone cleaning for 30 minutes. The thickness of the ITO film was 130 nm.

The glass substrate with a transparent electrode after cleaning was mounted on a substrate holder of a vacuum vapor deposition apparatus. First, Compounds HT-2 and HI-1 were co-deposited on the surface of the substrate on which the transparent electrode has been formed to cover the transparent electrode with the compounds, such that the proportion of Compound HI-1 was 3% by mass. Thus, a first hole-transporting layer having a thickness of 10 nm was formed.

On the first hole-transporting layer, Compound HT-2 was deposited to form a second hole-transporting layer having a film thickness of 80 nm.

On the second hole-transporting layer, Compound EBL-2 was deposited to form a third hole-transporting layer (also referred to as “electron barrier layer”) having a film thickness of 5 nm.

On the third hole-transporting layer, Compound BH-1 (host material) and Compound BD-2 (dopant material) were co-deposited such that the proportion of Compound BD-2 was 4% by mass, to form an emitting layer having a film thickness of 25 nm.

On the emitting layer, Compound 2-1 was deposited to form a first electron-transporting layer (also referred to as “hole barrier layer”) having a film thickness of 5 nm.

On the first electron-transporting layer, Compound ET-1 and Liq were co-deposited such that the proportion of Liq was 50% by mass, to form a second electron-transporting layer having a film thickness of 20 nm.

Metal Yb was deposited on the second electron-transporting layer to form an electron-injecting layer having a film thickness of 1 nm.

Metal Al was deposited on the electron-injecting layer to form a cathode having a film thickness of 50 nm.

The device configuration of the organic EL device of Example 2 is schematically described as follows:
ITO(130)/HT-2:HI-1(10:3%)/HT-2(80)/EBL-2(5)/BH-1:BD-2(25:4%)/Compound 2-1(5)/ET-1:Liq(20: 50%)/Yb(1)/Al(50)

Numerical values in parentheses indicate film thickness (unit: nm). In addition, the numerical values expressed as percentages in parentheses indicate the proportion (% by mass) of the latter compound in the corresponding layer.

### Example 3

An organic EL device was fabricated in the same manner as in Example 2, except that a compound described in Table 2 was used in place of Compound 2-1 for the formation of the first electron-transporting layer.

### <Evaluation 2 of Organic EL Device>

The external quantum efficiency and device lifetime of the organic EL devices fabricated in Examples 2 and 3 were evaluated as follows. The results are shown in Table 2.

### External Quantum Efficiency

A voltage was applied to the organic EL device such that the current density became 10 mA/cm², and EL emission spectrum was measured by a spectral radiance meter CS-2000 (manufactured by KONICA MINOLTA, INC.). The external quantum efficiency EQE (%) was calculated from the obtained spectral radiance spectrum.

### Device Lifetime

At room temperature, a voltage was applied to the organic EL device to be 50 mA/cm² in current density, and the time (LT95 (unit:hour) until the luminance becomes 95% of the initial luminance was measured.

TABLE 2

| | First electron-transporting layer | EQE (%) | LT95 (h) |
|---|---|---|---|
| Example 2 | Compound 2-1 | 10.5 | 151 |
| Example 3 | Compound 2-2 | 9.3 | 260 |
| Example 4 | Compound 2-3 | — | 165 |

It was found that the devices of Examples 2 and 3 had both superior EQE and LT95. It was found that the device of Example 4 was superior in LT95. Measurement of EQE was not conducted for the device of Example 4.

### <Synthesis of Compounds>

### (Synthesis Example 1) Synthesis of Compound 1-1

Compound 1-1 was synthesized in accordance with the synthetic route described below.

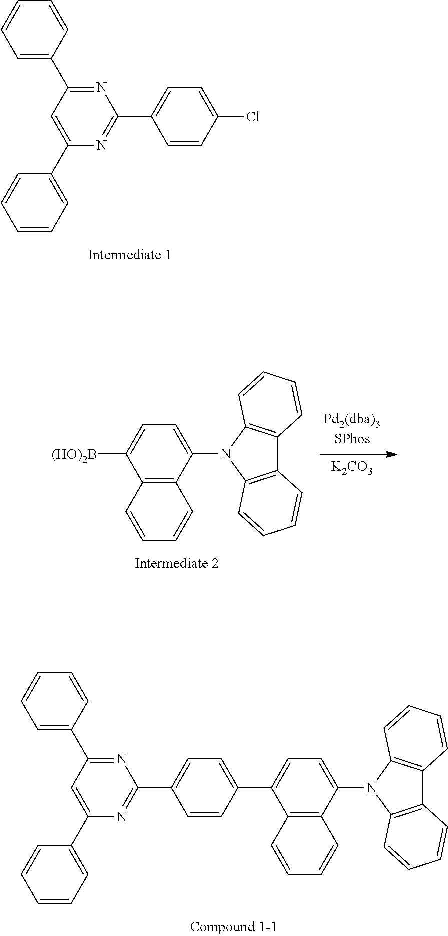

Under an argon atmosphere, to Intermediate 1 (3.00 g), Intermediate 2 (3.10 g), Pd₂(dba)₃ (160 mg), SPhos (287 mg), and potassium carbonate (2.42 g), dioxane (30 mL) and water (5 mL) were added and the mixture was refluxed for 6 hours. After completion of the reaction, the reaction liquid was subjected to short-pass silica gel column chromatography to concentrate the solvent. The resulting oil was recrystallized with hexane/toluene solvent to obtain white solid (4.6 g). The solid was purified by column chromatography to obtain Compound 1-1(3.7 g, yield: 71%) as the intended product. The result of mass spectrometric analysis was: m/e=600 for a molecular weight of 599.74.

### (Synthesis Example 2) Synthesis of Compound 2-1

Compound 2-1 was synthesized in accordance with the synthetic route described below.

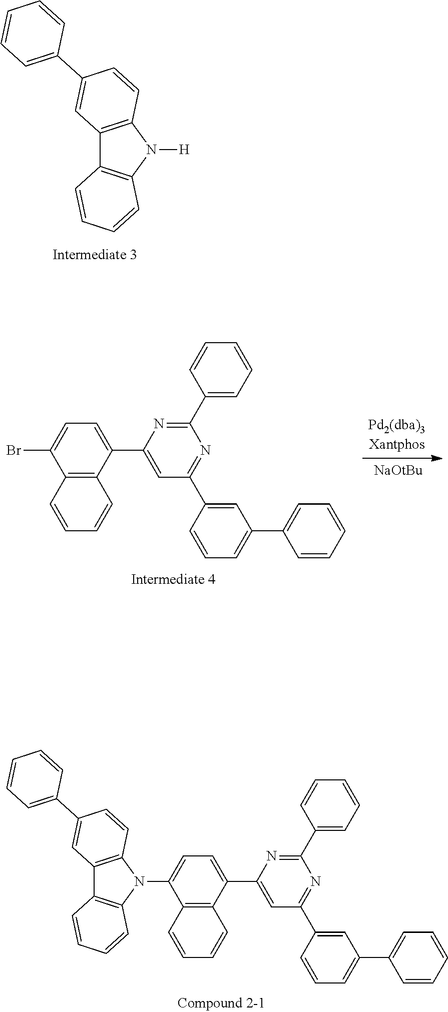 [SMILES: c1ccc(-c2ccc3[nH]c4ccccc4c3c2)cc1 ; Brc1ccc(-c2cc(-c3cccc(-c4ccccc4)c3)nc(-c3ccccc3)n2)c2ccccc12 ; c1ccc(-c2cccc(-c3cc(-c4ccc(-n5c6ccccc6c6cc(-c7ccccc7)ccc65)c5ccccc45)nc(-c4ccccc4)n3)c2)cc1 ; "NaOtBu"]

Under an argon atmosphere, to Intermediate 3 (1.46 g), Intermediate 4 (3.00 g), Pd₂(dba)₃ (165 mg), Xantphos (406 mg), and NaOtBu (1.28 g), xylene 30 mL was added, and the mixture was refluxed with heat for 5 hours. After completion of the reaction, the reaction liquid was purified by column chromatography to obtain Compound 2-1(2.4 g, yield: 61%) as the intended product. The result of mass spectrometric analysis was: m/e=676 for a molecular weight of 675.84.

### (Synthesis Example 3) Synthesis of Compound 2-2

Compound 2-2 was synthesized in accordance with the synthetic route described below.

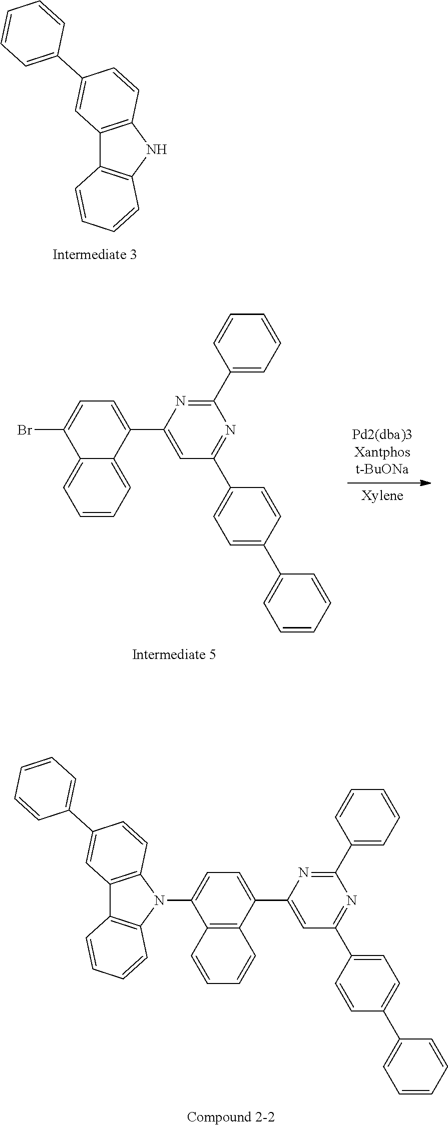 [SMILES: c1ccc(-c2ccc3[nH]c4ccccc4c3c2)cc1 ; Brc1ccc(-c2cc(-c3ccc(-c4ccccc4)cc3)nc(-c3ccccc3)n2)c2ccccc12 ; c1ccc(-c2ccc(-c3cc(-c4ccc(-n5c6ccccc6c6cc(-c7ccccc7)ccc65)c5ccccc45)nc(-c4ccccc4)n3)cc2)cc1]

Compound 2-2 was synthesised in the same manner as in Synthesis of Compound 2-1, except that Intermediate 5 was used in place of Intermediate 4 to obtain Compound 2-2 as white solids (9.0 g, yield: 76%). The result of mass spectrometric analysis was: m/e=676 for a molecular weight of 675.84, so that the obtained product was identified as the intended product.

### (Synthesis Example 4) Synthesis of Compound 2-3

Compound 2-3 was synthesized in accordance with the synthetic route described below.

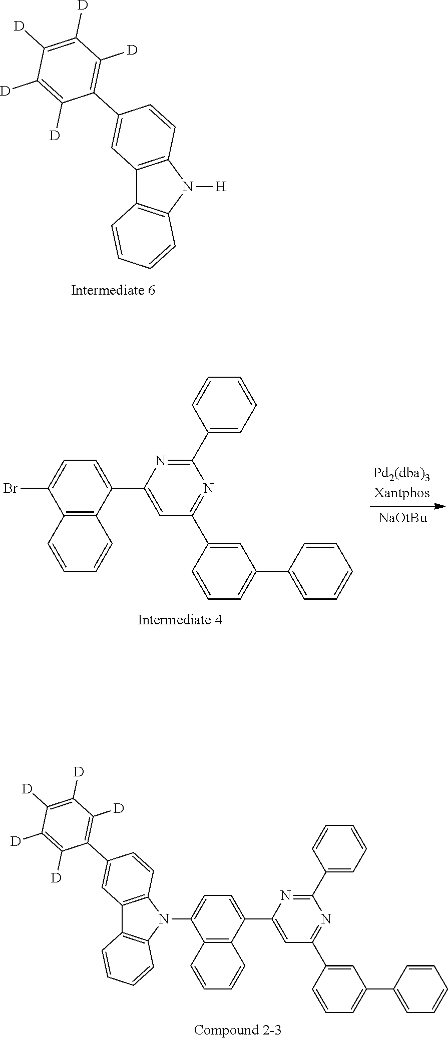 [SMILES: [2H]c1c([2H])c([2H])c(-c2ccc3[nH]c4ccccc4c3c2)c([2H])c1[2H] ; Brc1ccc(-c2cc(-c3cccc(-c4ccccc4)c3)nc(-c3ccccc3)n2)c2ccccc12 ; [2H]c1c([2H])c([2H])c(-c2ccc3c(c2)c2ccccc2n3-c2ccc(-c3cc(-c4cccc(-c5ccccc5)c4)nc(-c4ccccc4)n3)c3ccccc23)c([2H])c1[2H] ; "NaOtBu"]

Under an argon atmosphere, to Intermediate 6 (1.00 g), Intermediate 4 (2.00 g), Pd₂(dba)₃ (110 mg), Xantphos (271 mg), and NaOtBu (853 mg), xylene 20 mL was added, and the mixture was refluxed with heat for 5 hours. After completion of the reaction, the reaction liquid was purified by column chromatography to obtain Compound 2-3 (1.7 g, yield: 65%), which was an intended product. The result of mass spectrometric analysis was: m/e=681 for a molecular weight of 680.9.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification and the specification of Japanese application(s) on the basis of which the present application claims Paris convention priority are incorporated herein by reference in its entirety.

1. A compound represented by the following formula (101): 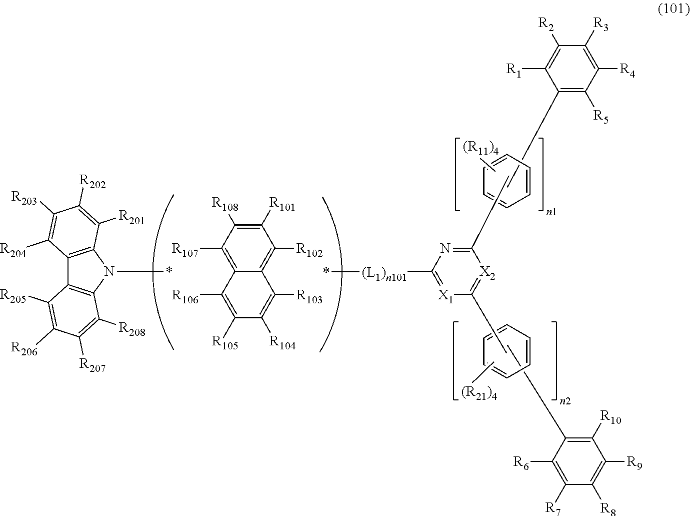
wherein in the formula (101),
one of X₁ and X₂ is N and the other is CH;
R₁ to R₁₀ are independently a hydrogen atom or a substituent R;
sets of adjacent two or more of R₁ to R₅ do not bond with each other;
sets of adjacent two or more of R₆ to R₁₀ do not bond with each other;
n1 is an integer of 0 to 3;
when n1 is 0, the structure in parentheses with n1 is a single bond;
when n1 is 2 or 3, the structures in parentheses with n1 may be the same as or different from each other;
R₁₁ is a hydrogen atom or a substituent R;
four R₁₁'s may be the same as or different from each other;
sets of adjacent two or more of four R₁₁'s do not bond with each other;
n2 is an integer of 0 to 3;
when n2 is 0, the structure in parentheses with n2 is a single bond;
when n2 is 2 or 3, the structures in parentheses with n2 may be the same as or different from each other;
R₂₁ is a hydrogen atom or a substituent R;
four R₂₁'s may be the same as or different from each other;
sets of adjacent two or more of four R₂₁'s do not bond with each other;
n101 is 0 or 1;
when n101 is 0, the structure in parentheses regarding n101 is a single bond;
L₁ is a substituted or unsubstituted phenylene group, or a substituted or unsubstituted naphthylene group;
one of R₁₀₁ to R₁₀₈ represents a single bond with (L₁) n101;
one of R₁₀₁ to R₁₀₈ which does not represent the single bond with (L₁)ₙ₁₀₁ represents a single bond with the nitrogen atom of the carbazole structure;
R₁₀₁ to R₁₀₈ which do not represent the single bonds with (L₁)ₙ₁₀₁ and with the nitrogen atom of the carbazole structure are independently a hydrogen atom or a substituent R;
sets of adjacent two or more of R₁₀₁ to R₁₀₈ do not bond with each other;
one or more sets of adjacent two or more of R₂₀₁ to R₂₀₈ form a substituted or unsubstituted aromatic hydrocarbon ring by bonding with each other, or do not bond with each other;
R₂₀₁ to R₂₀₈ which do not bond with each other are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;
when two or more substituent R's are present, the two or more substituent R's may be the same as or different from each other;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ may be the same as or different from each other.

2. The compound according to claim 1, wherein the compound represented by the formula (101) is a compound represented by the following formula (1): 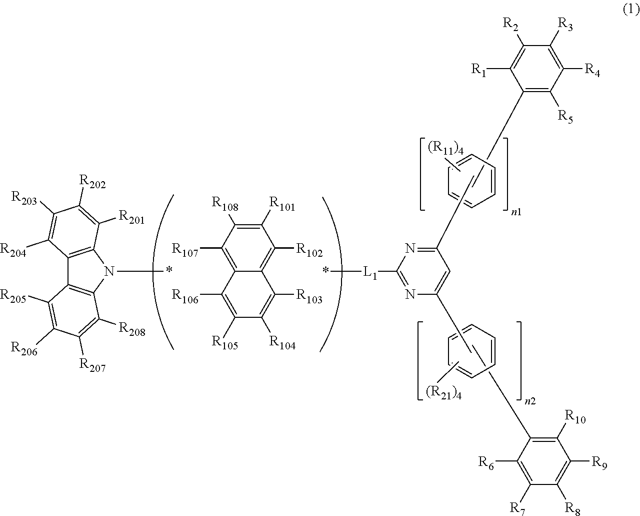
wherein in the formula (1),
R₁ to R₁₀ are independently a hydrogen atom or a substituent R;
sets of adjacent two or more of R₁ to R₅ do not bond with each other;
sets of adjacent two or more of R₆ to R₁₀ do not bond with each other;
n1 is an integer of 0 to 3;
when n1 is 0, the structure in parentheses with n1 is a single bond;
when n1 is 2 or 3, the structures in parentheses with n1 may be the same as or different from each other;
R₁₁ is a hydrogen atom or a substituent R;
four R₁₁'s may be the same as or different from each other;
sets of adjacent two or more of four R₁₁'s do not bond with each other;
n2 is an integer of 0 to 3;
when n2 is 0, the structure in parentheses with n2 is a single bond;
when n2 is 2 or 3, the structures in parentheses with n2 may be the same as or different from each other;
R₂₁ is a hydrogen atom or a substituent R;
four R₂₁'s may be the same as or different from each other;
sets of adjacent two or more of four R₂₁'s do not bond with each other;
L₁ is a substituted or unsubstituted phenylene group, or a substituted or unsubstituted naphthylene group;
one of R₁₀₁ to R₁₀₈ represents a single bond with L₁;
one of R₁₀₁ to R₁₀₈ which does not represent the single bond with L₁ represents a single bond with the nitrogen atom of the carbazole structure;
R₁₀₁ to R₁₀₈ which do not represent the single bonds with L₁ and with the nitrogen atom of the carbazole structure are independently a hydrogen atom or a substituent R;
sets of adjacent two or more of R₁₀₁ to R₁₀₈ do not bond with each other;
one or more sets of adjacent two or more of R₂₀₁ to R₂₀₈ form a substituted or unsubstituted aromatic hydrocarbon ring by bonding with each other, or do not bond with each other;
R₂₀₁ to R₂₀₈ which do not bond with each other are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;
when two or more substituent R's are present, the two or more substituent R's may be the same as or different from each other;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ may be the same as or different from each other.

3. The compound according to claim 1, wherein R₁₀₂ represents a single bond with L₁, and R₁₀₇ represents a single bond with the nitrogen atom of the carbazole structure.

4. The compound according to claim 1, wherein one or more sets of adjacent two or more of R₂₀₁ to R₂₀₈ do not bond with each other.

5. The compound according to claim 1, wherein L₁ is a substituted or unsubstituted phenylene group.

6. The compound according to claim 2, wherein the compound represented by the formula (1) is a compound represented by the following formula (11): 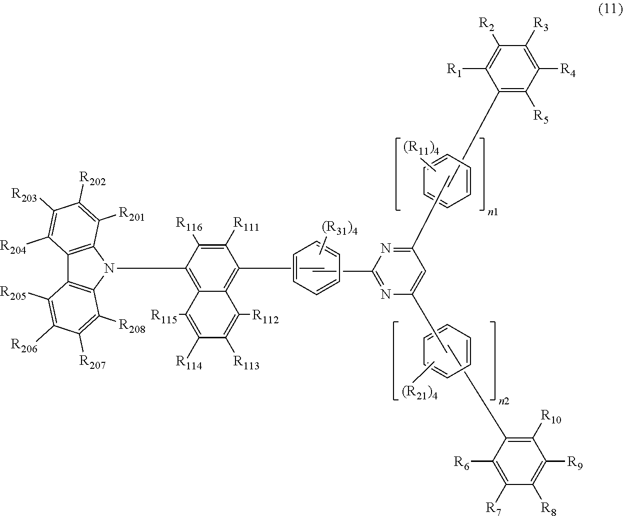
wherein in the formula (11),
R₁ to R₁₀, R₁₁, R₂₁, n1, n2, and R₂₀₁ to R₂₀₈ are as defined in the formula (1);
R₃₁ is a hydrogen atom or a substituent R;
four R₃₁'s may be the same as or different from each other;
sets of adjacent two or more of four R₃₁'s do not bond with each other;
R₁₁₁ to R₁₁₆ are independently a hydrogen atom or a substituent R;
sets of adjacent two or more of R₁₁₁ to R₁₁₆ do not bond with each other, and
the substituent R is as defined in the formula (1).

7. The compound according to claim 1, wherein n1 and n2 are 0.

8. The compound according to claim 1, wherein R₁ to R₁₀ are hydrogen atoms.

9. The compound according to claim 1, wherein R₂₀₁ to R₂₀₈ are hydrogen atoms.

10. The compound according to claim 2, wherein R₁₀₁ to R₁₀₈ in the formula (1) which do not represent a single bond or Rin to R₁₁₆ in the formula (11) which do not represent a single bond are hydrogen atoms.

11. The compound according to claim 1, wherein the compound represented by the formula (101) is a compound represented by the following formula (2): 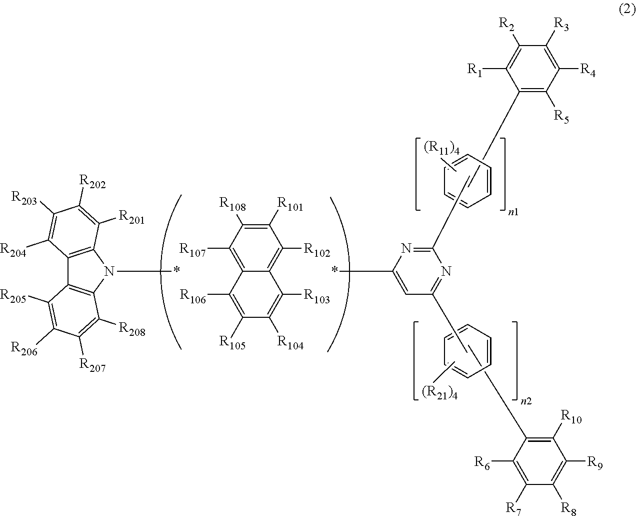
wherein in the formula (2),
R₁ to R₁₀ are independently a hydrogen atom or a substituent R;
sets of adjacent two or more of R₁ to R₅ do not bond with each other;
sets of adjacent two or more of R₆ to R₁₀ do not bond with each other;
n1 is an integer of 0 to 3;
when n1 is 0, the structure in parentheses with n1 is a single bond;
when n1 is 2 or 3, the structures in parentheses with n1 may be the same as or different from each other;
R₁₁ is a hydrogen atom or a substituent R;
four R₁₁'s may be the same as or different from each other;
sets of adjacent two or more of four R₁₁'s do not bond with each other;
a pair of R₁ and R₁₁ do not bond with each other;
a pair of R₅ and R₁₁ do not bond with each other;
n2 is an integer of 1 to 3;
when n2 is 2 or 3, the structures in parentheses with n2 may be the same as or different from each other;
R₂₁ is a hydrogen atom or a substituent R;
four R₂₁'s may be the same as or different from each other;
sets of adjacent two or more of four R₂₁'s do not bond with each other;
a pair of R₆ and R₂₁ do not bond with each other;
a pair of R₁₀ and R₂₁ do not bond with each other;
one of R₁₀₁ to R₁₀₈ represents a single bond with the carbon atom of the pyrimidine structure;
one of R₁₀₁ to R₁₀₈ which does not represent a single bond with the carbon atom of the pyrimidine structure represents a single bond with the nitrogen atom of the carbazole structure;
R₁₀₁ to R₁₀₈ which do not represent the single bonds with the carbon atom of the pyrimidine structure and with the nitrogen atom of the carbazole structure are independently a hydrogen atom or a substituent R;
sets of adjacent two or more of R₁₀₁ to R₁₀₈ do not bond with each other;
one or more sets of adjacent two or more of R₂₀₁ to R₂₀₈ form a substituted or unsubstituted aromatic hydrocarbon ring by bonding with each other, or do not bond with each other;
R₂₀₁ to R₂₀₈ which do not bond with each other are independently a hydrogen atom or a substituent R;
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms;
when two or more substituent R's are present, the two or more substituent R's may be the same as or different from each other;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ may be the same as or different from each other.

12. The compound according to claim 11, wherein R₁₀₂ represents a single bond with the carbon atom of the pyrimidine structure, and R₁₀₇ represents a single bond with the nitrogen atom of the carbazole structure.

13. The compound according to claim 11, wherein one or more sets of adjacent two or more of R₂₀₁ to R₂₀₈ do not bond with each other.

14. The compound according to claim 11, wherein the compound represented by the formula (2) is a compound represented by the following formula (21): 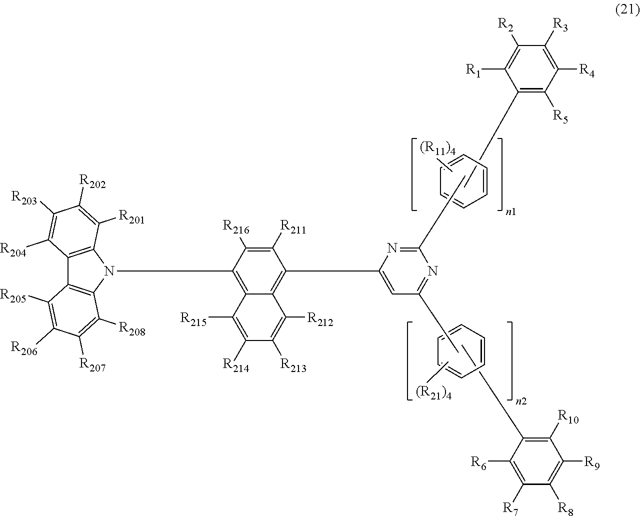
wherein in the formula (21),
R₁ to R₁₀, R₁₁, R₂₁, n1, n2, and R₂₀₁ to R₂₀₈ are as defined in the formula (2);
R₂₁₁ to R₂₁₆ are independently a hydrogen atom or a substituent R;
sets of adjacent two or more of R₂₁₁ to R₂₁₆ do not bond with each other, and
the substituent R is as defined in the formula (2).

15. The compound according to claim 11, wherein n1 is 0.

16. The compound according to claim 11, wherein R₁ to R₁₀ are hydrogen atoms.

17. The compound according to claim 11, wherein any of R₂₀₁ to R₂₀₈ is a substituted or unsubstituted aryl group including 6 to 50 ring carbon atoms,
the rest of R₂₀₁ to R₂₀₈ are independently selected from the group consisting of
a hydrogen atom,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
Halogen atom, cyano group, nitro group, and
a substituted or unsubstituted monovalent heterocyclic group including 5 to 50 ring atoms.

18. The compound according to claim 11, wherein R₂₀₃ is a substituted or unsubstituted aryl group including 1 to 50 ring carbon atoms.

19. The compound according to claim 11, wherein R₂₀₃ is a substituted or unsubstituted phenyl group.

20. The compound according to claim 11, wherein R₂₀₁, R₂₀₂, and R₂₀₄ to R₂₀₈ are hydrogen atoms.

21. The compound according to claim 11, wherein R₂₁₁ to R₂₁₆ are hydrogen atoms.

22. The compound according to claim 1, wherein the substituent in the case of “substituted or unsubstituted”, and the substituent R are independently a group selected from the group consisting of
an alkyl group including 1 to 50 carbon atoms,
an aryl group including 6 to 50 ring carbon atoms, and
a monovalent heterocyclic group including 5 to 50 ring atoms.

23. The compound according to claim 1, wherein the substituent in the case of “substituted or unsubstituted”, and the substituent R are independently a group selected from the group consisting of
an alkyl group including 1 to 18 carbon atoms,
an aryl group including 6 to 18 ring carbon atoms, and
a monovalent heterocyclic group including 5 to 18 ring atoms.

24. The compound according to claim 1, wherein the compound is a material for an organic electroluminescence device.

25. An organic electroluminescence device comprising: a cathode;
an anode; and
one or two or more organic layers arranged between the cathode and the anode,
wherein at least one of the organic layers comprises the compound according to claim 1.

26. The organic electroluminescence device according to claim 25, which comprises an anode, an emitting layer, an electron-transporting region, and a cathode in this order, wherein
the electron-transporting region comprises the compound.

27. The organic electroluminescence device according to claim 26, wherein the electron-transporting region comprises a first layer and a second layer in this order from the emitting layer side, and
the first layer comprises the compound.

28. An electronic apparatus equipped with the organic electroluminescence device according to claim 24.
